(19) United States
(12) Patent Application Publication
(10) Pub. No.: US 2019/0227055 A1
(43) Pub. Date: Jul. 25, 2019

(54) Compositions and Methods for Photocleavage Based Concentration and/or Purification of Analytes

(71) Applicant: AmberGen, Inc., Watertown, MA (US)

(72) Inventors: Mark J. Lim, Reading, MA (US); Heather P. Ostendorff, Framingham, MA (US); Zhi Wan, West Roxbury, MA (US); John Gillespie, Dover, MA (US); Kenneth J. Rothschild, Newton, MA (US)

(21) Appl. No.: 16/252,378

(22) Filed: Jan. 18, 2019

Related U.S. Application Data
(60) Provisional application No. 62/619,287, filed on Jan. 19, 2018.

(51) Int. Cl. G01N 33/53; B01L 3/00; G01N 33/543; G01N 21/64

(52) U.S. Cl. CPC G01N 33/5304; B01L 3/5085; B01L 3/52; G01N 33/54386; G01N 2021/6439; G01N 21/6452; B01L 2300/12; B01L 2300/123; G01N 21/6428

(57) ABSTRACT

The invention relates to compositions and methods for the concentration and/or purification of analytes, such as biomarkers, typically from complex biological samples such as whole blood, serum or plasma. This invention also relates to the use of binding agents, such as antibodies, aptamers, antigens and engineered protein scaffold based binding agents (e.g. commercially available Affibodies®), to facilitate the concentration and/or purification of said analytes. This invention further relates to assays used to detect, measure and/or quantify the analyte after its concentration and/or purification, preferably solid-phase immunoassays and more preferably multiplex solid-phase immunoassays.

This invention was made with government support under R44AI100424 awarded by the National Institutes of Health. The government has certain rights in this invention.

### FIELD OF THE INVENTION

The field of this invention relates to compositions and methods for the concentration and/or purification of analytes, such as biomarkers, typically from complex biological samples such as whole blood, serum or plasma. This invention also relates to the use of binding agents, such as antibodies, aptamers, antigens and engineered protein scaffold based binding agents (e.g. commercially available AfFibodies®), to facilitate the concentration and/or purification of said analytes. Furthermore, photocleavable chemical linkers are used to attach the binding agents to a substrate so that analytes may be captured and then photo-released in a concentrated and/or purified form. This invention further relates to the types of substrates used, such as beads or microtiter/microwell plates. This invention further relates to assays used to detect, measure and/or quantify the analyte after its concentration and/or purification, preferably solid-phase immunoassays and more preferably multiplex solid-phase immunoassays. Said assays are typically used in the field of diagnostics, prognostics, disease monitoring and guiding therapies. Examples of the utility of this invention are in the fields of serological detection of allergen-specific IgE (sIgE) in the diagnosis of allergies, detection of circulating tumor proteins in the diagnosis of cancer and detection of antibodies to Human Leukocyte Antigens (HLA) in the prevention and diagnosis of rejection in tissue/organ transplants and blood transfusions. In a preferred embodiment, purification of the analyte is necessary to eliminate interference from the biological sample matrix with the subsequent detection, measurement and/or quantification of said analyte. This interference is most commonly referred to as the “matrix effect”. In another preferred embodiment, concentration of the analyte is necessary to facilitate its detection, measurement and/or quantification.

### BACKGROUND OF THE INVENTION

An analyte is any molecule or biomolecule to be detected, measured and/or quantified. Biomarkers, a class of analyte, include molecules or biomolecules such as proteins or DNA which are indicative of, for example, a disease or disease stale/stage, or indicative of response to therapy or the probability of response to therapy. As addressed by the present invention, the ability to efficiently and gently concentrate and/or purify biomarkers, in a simple and effective manner, is important for both highly sensitive and quantitatively accurate biomarker detection/measurement. This ability is also necessary to facilitate clinical diagnostic applications where reproducibility, sensitivity and quantitative accuracy are important considerations.

### The Problem of the Matrix Effect in Biomarker Detection

Solid-phase immunoassays such as the enzyme linked immunosorbent assay (ELISA) and the fluorescence enzyme immunoassay (FEIA) have been a mainstay in biomarker detection and immunodiagnostics for decades. However, emerging multiplex assays, that is, assays which simultaneously measure multiple biomarkers in a single experiment using single reaction vessel, promise significant advantages such as reduced sample volume required, higher throughput and lower cost per biomarker. A variety of solid-phase immunoassay platforms have been developed to meet the needs for multiplex or multi-biomarker detection. Mainstream platforms include those based on microarrays (e.g. MSD MultSpot® technology [Kenten, Davydov et al. (2005) Methods Enzymol 399; 682-701]), microfluidics (e.g. ProteinSimple® Simple Plex™ assay using hollow glass microfluidic assay channels [Leligdowicz, Conroy et al. (2017) PLoS One 12; e0175130]) and microspheres (e.g. Luminex® xMAP® platform using microspheres encoded with fluorophores [Fulton, McDade et al. (1997) Clin Chem 43: 1749-56]).

Although these systems have been somewhat useful for basic research, they have generally failed to transition into the clinic [Tighe, Ryder et al. (2015) Proteomics Clin Appl 9: 406-22], in large part due to the well-known “matrix effect”. This effect is caused by the presence of non-target constituents in complex biological samples such as blood which interfere with detection/measurement/quantification of the target biomarkers.

Importantly, while all assays suffer from the matrix effect, multiplex assays are especially susceptible compared to conventional non-multiplex assays such as ELISA [Martins, Pasi et al. (2004) Clin Diagn Lab Immunol 11:325-9; Dias, Van Doren et al. (2005) Clin Diagn Lab Immunol 12: 959-69; Waterboer, Sehr et al. (2006) J Immunol Methods 309: 200-4; de Jager, Bourcier et al. (2009) BMC immunology 10: 52; Chiu, Lawi et al. (2010) JALA 15: 233-42; Churchman, Geiler et al. (2012) Clinical and experimental rheumatology 30: 534-42; Rosenberg-Hasson, Hansmann et al. (2014) Immunol Res 58: 224-33]. This is in large part because the necessary miniaturization of these assays (e.g. microarray, microfluidic or microsphere formats) results in a very low binding capacity of the assay surfaces. Thus, contaminants can more easily saturate the assay surface compared to conventional non-multiplex assays. Interference can be caused by a variety of mechanisms (FIG. 1.1-1.4B) including: i) low specificity heterophile antibodies that bridge proteins on the assay surface such as the assay capture antibody, with the detection antibodies in immunoassays, yielding a false positive signal (FIG. 1.2); ii) matrix-induced microsphere aggregation (e.g. with the Luminex® immunoassay platform) via heterophilic antibodies or other bound non-target agents (FIG. 1.3); and iii) specific or non-specific binding of non-target matrix constituents to any component of the assay, which can either suppress assay signal (FIG. 1.4a) or mediate background (FIG. 1.4b). Note that while a sandwich immunoassay is shown in FIG. 1.1-1.4B (with a capture antibody on the assay surface), other immunoassay formats include where an antigen (e.g. allergen) is on the assay surface as the capture agent (typically to capture antibody analytes/biomarkers such as allergen-specific IgE [sIgE] for example). Regardless, the matrix effects are similar. In addition to the matrix effects shown in FIG. 1.1-1.4B, high viscosity of the sample matrix or undesirable sample conductance can interfere with the microfluidics commonly used for multiplex assays and miniaturized parallelized assays [Chiu, Lawi et al. (2010) JALA 15: 233-42; Stern, Vacic et al. (2010) Nat Nanotechnol 5: 138-42]. Overall, the matrix effect degrades not only the sensitivity but also the dynamic range, quantitative accuracy and reproducibility of multiplex assays [Martins, Pasi et al. (2004) Clin Diagn Lab Immunol 11: 325-9; Dias, Van Doren et al. (2005) Clin Diagn Lab Immunol 12: 959-69; Waterboer, Sehr et al. (2006) J Immunol Methods 309: 200-4; de Jager, Bourcier et al. (2009) BMC immunology 10: 52; Chiu, Lawi el al. (2010) JALA 15: 233-42; Churchman, Geiler et al. (2012) Clinical and experimental rheumatology 30: 534-42; Rosenberg-Hasson, Hansmann et al. (2014) Immunol Res 58: 224-33]. As such, multiplex assays generally fail to match the robust performance of their industry-standard non-multiplex counterparts such as ELISA.

The problem of the matrix effect in multiplex assays is illustrated in one report evaluating an immobilized-antigen assay for HPV using the Luminex® microsphere platform [Dias, Van Doren et al. (2005) Clin Diagn Lab Immunol 12: 959-69], “Because sera from naturally infected individuals typically have very low concentrations of antibodies to HPV virions, the sera must be tested at a high concentration. This challenge is compounded by the fact that at high concentrations there are considerable matrix effects caused by interfering substances in serum that vary by individual. These interfering substances can include lipids, cholesterol, proteins, and heterophilic antibodies.”

### Additional Matrix Effects in the Serological Detection of Antibodies

In many cases, it is advantageous to detect specific immunoglobulin (antibody) classes (isotypes) or subclasses (subtypes) from a serum or plasma sample for diagnostic purposes. In mammals, there exist five main classes of immunoglobulin: IgG, IgD, IgA, IgE and IgM. IgG exists at the highest concentration in human serum, representing 70-85% of the total immunoglobins. In addition, there are four subclasses of IgG (IgG1, IgG2, IgG3 and IgG4). In comparison, IgD accounts for 1%, IgM (5-10%), IgA (5-10%) and IgE under 1% [Collins, Tsui et al. (2002) Eur J Immunol 32: 1802-10; Cruse and Lewis (Atlas of Immunology, CRC Press/Taylor & Francis, Boca Raton, FLa., 2010)]. In many cases, different types of antibodies may compete for the same antigen that is incorporated into an immunoassay surface used for detection, such surfaces including microspheres that comprise part of a multiplex assay. This cross-talk of different antibody species can contribute to the matrix effect. For example, IgG which is at much higher concentration in human serum compared to IgE, can effectively mask antigens and thus lower the effective measurement of allergen-specific IgEs in the diagnosis of allergies. This is especially true of the IgG4 subclass which is believed to moderate in many cases the allergic response [Rispens, Derksen et al. PLoS One 8: e55566; Hofman (1995) Rocz Akad Med Bialymst 40: 468-73; Visco, Dolecek et al. (1996) J Immunol 157: 956-62; Kadooka, Idota et al. (2000) Int Arch Allergy Immunol 122: 264-9; Jarvinen, Chatchatee et al. (2001) Int Arch Allergy Immunol 126: 111-8; Shreffler, Lencer et al. (2005) J Allergy Clin Immunol 116: 893-9; Stapel, Asero et al. (2008) Allergy 63: 793-6; Carr, Chan et al. (2012) Allergy Asthma Clin Immunol 8: 12; Guhsl, Hofstetter et al. (2015) Allergy 70: 59-66]. In another example, detection of IgG antibodies to Human Leukocyte Antigens (HLA) is used in the prevention or diagnosis of rejection in tissue/organ transplants and blood transfusions. However, specific matrix effects have been observed in the immunoassay-based detection of these antibodies, including interference from competing IgM antibodies, or masking of the IgG by bound complement [Kosmoliaptsis, Bradley et al. (2009) Transplantation 87: 813-20; Carey, Boswijk et al. (2016) Transpl Immunol 37: 23-7]. Finally, detection of virus-specific IgM antibodies is important in the diagnosis of infectious diseases. IgM detection is especially important when the viremic phase is short (e.g. with Zika), precluding the nucleic acid based detection of a virus in many cases once this phase has passed. IgM is also important to distinguish an older and potentially previous infection (IgG), from an active/acute-phase infection (IgM) [Landry (2016) Clin Vaccine Immunol 23: 540-5], yet the presence of competing IgGs can interfere with the detection of the IgMs.

### The Problem of Low Biomarker Abundance

Compounding the problem of the matrix effect is that most useful biomarkers are typically in low abundance in the biological sample. This is exemplified in blood-based cancer and allergy testing as discussed below:

In the example of cancer diagnostics, the most highly specific blood-based protein biomarkers are those directly shed from the tumor, instead of indirect measures such as biomarkers of inflammatory host-response to the tumor (e.g. cytokines) which can also occur in a variety of non-cancerous conditions [Tang, Beer et al. (2012) J Proteome Res 11: 678-91; Beer, Wang et al. (2013) PLoS One 8: e60129]. However, by the very nature that these tumor-shed biomarkers are diluted from a distal site into the general circulation, they will be present at extremely low concentrations in comparison to a variety of far more abundant blood proteins and other biomolecules [Rusling, Kumar et al. (2010) Analyst 135: 2496-511; Hori and Gambhir (2011) Sci Transl Med 3: 109ra116; Tang, Beer et al. (2012) J Proteome Res 11: 678-91; Beer, Wang et al. (2013) PLoS One 8: e60129; Konforte and Diamandis (2013) Clin Chem 59: 35-7]. Thus, not surprisingly, at the biomarker discovery stage, model experimental systems are often used in which the biomarkers are “easier” to detect (e.g. systems where biomarkers are at higher relative abundance). Examples include analyzing the tumor tissue itself, cell culture supernatants and tumor xenograft models where biomarkers are present or shed at high concentration [Pitteri, JeBailey et al. (2009) PLoS One 4: e7916; Tang, Beer et al. (2012) J Proteome Res 11: 678-91; Beer, Wang et al. (2013) PLoS One 8: e60129; Birse, Lagier et al. (2015) Clin Proteomics 12: 18]. However, the subsequent validation and clinical assay of tumor-shed biomarkers needs to be done on actual human serum for early-stage cancer detection (when the disease is most curable), and therefore the aforementioned model experimental systems ultimately do not solve the problem of low biomarker abundance (or the aforementioned matrix effect).

In the example of blood-based allergy diagnostics, where allergens are immobilized on an assay surface to bind and detect allergen-specific IgE (sIgE) antibodies from the patient, it is important to consider that IgE is the lowest abundance immunoglobulin in human blood, approximately 270,000-fold less abundant than IgG and 71,000-fold less abundant than IgA [Golub and Green (1991) Immunology: A Synthesis, 2nd Edition, Publisher: Sinauer Associates, Inc.: Chapter 6, pg, 95]. This low abundance problem is compounded by the fact that in addition to the aforementioned generic matrix effects (e.g. FIG. 1.1-1.4B), allergy assays can be further compromised by non-IgE allergen-specific antibodies present in the blood which also bind (and saturate) the allergen (antigen) on the immunoassay surface. For example, allergen-specific immunoglobulins of other classes including IgG and IgA may be induced (same epitopes) but are not recommended for diagnostic testing as only IgE is responsible for the immediate-type hypersensitivity reactions [Rispens, Derksen et al. PLoS One 8: e55566; Hofman (1995) Rocz Akad Med Bialymst 40: 468-73; Visco, Dolecek et al. (1996) J Immunol 157; 956-62; Kadooka, Idota et al. (2000) Int Arch Allergy Immunol 122; 264-9; Jarvinen, Chatchatee et al. (2001) Int Arch Allergy Immunol 126; 111-8; Shreffler, Lencer et al. (2005) J Allergy Clin Immunol 16: 893-9; Stapel, Asero et al. (2008) Allergy 63: 793-6; Carr, Chan et al. (2012) Allergy Asthma Clin Immunol 8: 12; Guhsl, Hofstetter et al. (2015) Allergy 70; 59-66]. This problem of low-abundance IgE and competing high abundance immunoglobulins of other types is even further exacerbated since the standard practice (in food allergy testing for example) is to use whole food extracts as the antigen (allergen) on the immunoassay surface (since not all allergenic proteins have been identified). Since whole food extracts can contain hundreds to thousands of proteins, many of which are irrelevant (not allergens), the amount of actual available allergen and hence the surface binding capacity for allergen-specific IgE (sIgE) is very low. This is especially the case for multiplex immunoassay platforms where the capacity of the assay surface is small to begin with (as discussed earlier).

### SUMMARY OF THE INVENTION

This invention relates to compositions and methods of use of binding agents directly or indirectly attached to substrates by a photocleavable linker. This invention also relates to methods of using said compositions to capture/isolate and then photo-release analytes, such as biomarkers, for the purpose of concentrating and/or purifying said analytes from a sample (a process hereafter referred to as PC-PURE). In a preferred embodiment, the concentrating and/or purifying of said analytes is useful for the purpose of improved detection/measurement/quantification of said analytes, for example using a solid-phase immunoassay, such as to aid in the diagnosis of disease.

Preferred binding agents include, but are not limited to, antibodies, aptamers, antigens and engineered protein scaffold based binding agents (e.g. commercially available Affibodies®).

Preferred substrate types include, but are not limited to, microtiter plates (alternatively referred to as multi-well or microwell plates, or microplates), for example 6-, 12-, 24-, 96-, 384- and 1,536-well plates, having wells comprised of, but not limited to, any one of the following materials or any combination thereof (to which binding agents are directly or indirectly attached by a photocleavable linker): polymers; plastics; glass. Additional preferred substrate materials include high capacity 3-dimensional porous matrices such as agarose, polyacrylamide and PEG based films, gels and beads; and porous membranes (e.g. micro-porous, that is, having micron-scale pores) such as nitrocellulose (cellulose nitrate), cellulose acetate and/or polyvinylidene fluoride (PVDF). These additional substrate materials, to which binding agents are directly or indirectly attached by a photocleavable linker, may coat or form the bottoms of the microtiter plate wells, for example. As described in the Detailed Description of Invention, microtiter plates are to be distinguished from microarrays, whereby microarrays are not suitable for the concentrating and/or purifying analytes from samples as described in the present invention.

Analyte concentration and/or purification is typically from complex biological samples such as whole blood, serum or plasma. In a preferred embodiment, purification of the analyte is necessary to eliminate interference from the non-target constituents in complex biological samples with the detection, measurement and/or quantification of the analyte. This interference is most commonly referred to as the “matrix effect”. In another preferred embodiment, concentration of the analyte is performed to facilitate downstream detection, measurement and/or quantification of the analyte, such as with low abundance analytes. In some embodiments, the binding agents attached to substrates by a photocleavable linker may also be conjugated to a detectable label, to facilitate downstream detection, measurement and/or quantification of the analyte by way of the binding agent. In one example of the utility of this invention, IgE is concentrated and/or purified from biological samples such as whole blood, serum or plasma prior to detection of allergen-specific IgE antibodies (sIgE) using subsequent immunoassays, as a method for in vitro diagnosis of allergies. In another preferred embodiment, circulating proteins shed from tumors are concentrated and/or purified and then detected, e.g. by immunoassay, for the diagnosis of cancer. Furthermore, in a preferred embodiment, concentrated/purified analytes are detected, measured and/or quantified using solid-phase immunoassays, more preferably multiplex solid-phase immunoassays. It is to be understood that the invention is not intended to be limited to any one particular analyte or class of analytes.

### DETAILED DESCRIPTION OF THE INVENTION

It is to be clearly understood that this invention is not limited to the particular compositions and methods described herein, as these may vary. It is also to be understood that the terminology used herein is for the purpose of describing particular embodiments only, and it is not intended to limit the scope of the present invention.

### The Basic Approach

U.S. Pat. No. 8,906,700 is hereby incorporated by reference in its entirety.

A simplified flow diagram for one embodiment of the present invention is shown in FIG. 2a (FIG. 2a and 2b not drawn to scale). In this embodiment, an analyte (e.g. biomarker) is concentrated and/or purified (by the PC-PURE process) from a whole blood, serum or plasma sample. This basic embodiment of the invention consists of several steps briefly described below and in more detail in the following sections (along with other embodiments):

Step 1. Collect Sample—In the example shown in FIG. 2a the sample is blood (e.g. collected by a finger-stick as depicted; a heel-stick or standard venipuncture can also be used). In the case of a blood sample, it may be used as whole blood or converted to serum or plasma.

Step 2. Capture Biomarker—The biomarker (a class of analyte) in the sample is then captured/isolated by a binding agent (aptamer depicted) which is immobilized on a substrate (the substrate type depicted is a well of a microtiter plate, which contains a porous membrane, gel or film as the substrate material). The binding agent is immobilized on the substrate by a photocleavable (PC) linker (together referred to as the “PC-Binding Agent” in FIG. 2a).

Step 3. Separate Sample from Captured Biomarker—The substrate is washed with a controlled buffer solution to remove non-target sample matrix constituents that would potentially interfere with the downstream detection, measurement and/or quantification of the biomarker.

Step 4. Photo-Release Biomarker—Illumination of the substrate with the appropriate wavelength and intensity of light photo-releases the [PC-Binding Agent]-[Biomarker] complex in concentrated and/or purified form. Note that as depicted in FIG. 2a, the input sample volume can be larger than the photo-release volume to facilitate concentrating of the analyte in addition to purification.

In one particular embodiment, following the PC-PURE process, the photo-released biomarker can be measured in a downstream multiplex immunoassay (see Steps 5-6 of FIG. 2b). In this case, the photo-released [PC-Binding Agent]-[Biomarker] complex is combined with suitable immunoassay surface (e.g. Luminex® microsphere surface for a multiplex assay in this case) which is coated with a second capture agent such as a capture antibody or antigen, to re-capture the biomarker (Step 5 of FIG. 2b). Detection in the assay can for example be achieved using a detection antibody (depicted in FIG. 2b; a reporter label such as a fluorophore is not shown). Alternatively, the PC-Binding Agent can be used also for detection in the assay (e.g. if bearing a detectable label; label not depicted in FIG. 2b). Assay readout is achieved in a companion instrument such as the Luminex® MagPix® reader for detection, measurement and/or quantification of the biomarker (Step 6 in FIG. 2b). It is to be understood that the invention is not intended to be limited to the above embodiment.

### Substrates for Immobilizing the Photocleavable Binding Agent

The present invention uses binding agents attached to a substrate through a photocleavable linker for the purposes of isolating, concentrating and/or purifying analytes, for example, biomarkers, from samples. The substrate can be a variety of types as detailed below.

In one preferred embodiment, the substrate type is a bead, microsphere or another type of particle as will be recognized by those skilled in the art of affinity isolation/separation.

Substrate types can also include the surfaces of reaction vessels or tubes (e.g. test tubes, blood collection tubes or micro-centrifuge tubes). Additional examples of substrate types include polymeric capsules, pellets or plugs. In one embodiment, capsules, pellets or plugs (e.g. made of porous materials) are those which can be placed into a reaction vessel or fitted into the end of a pipette tip (e.g. to form a micro-column or mini-column).

In one preferred embodiment, the substrate type is the well of a microtiter plate (e.g. 6-, 12-, 24-, 48-, 96-, 384- or 1,536-well plate; including solid plates or membrane-bottom filter plates; standard depth wells or deep-wells of various shapes including flat-bottom, U-bottom, V-bottom, pyramid-bottom or conical-bottom wells; strip-well plates whereby columns or rows of wells can be removed and processed separately are also included). These plates are alternatively referred to as multi-well or microwell plates, or microplates. Collectively, these plates are hereafter referred to as microtiter plates. The invention is not limited to commercially available microtiter plates since custom plates can be constructed for specialized applications.

A microtiter plate is a flat plate with multiple “wells” which serve in essence as small test tubes. The microtiter plate has become a standard tool in analytical research and clinical diagnostic testing laboratories. A very common usage is in the enzyme-linked immunosorbent assay (ELISA). Each well of a microtiter plate can contain a liquid or other material such as a gel or suspension of particles. A microtiter plate typically has 6, 12, 24, 48, 96, 384 or 1,536 sample wells arranged in a rectangular matrix, normally with the dimension of 128 mm×86 mm. Each well of a microtiter plate typically holds somewhere between tens of nanoliters to several milliliters of liquid. They can also be used to store dry powder or as racks to support tube inserts. In some cases, the wells can contain a dry filter material cut to fit the well dimension such as filters containing dried blood spots (DBS) which can later be exposed to a liquid to extract an analyte in the dried blood. Wells can be either circular or square and have flat, tapered, rounded, pyramidal or conical bottoms. The wells can possess on the inside surface various coatings of varying compositions and thickness including but not limited to polymers, gels, metal oxide and growth medium for cells. In some cases the coatings can be made monomolecularly thick such as sputtered metals like gold. Active molecules can be incorporated into the coatings including biologically active enzymes, capture molecules such as streptavidin, antibodies or aptamers, nucleic acids, carbohydrates and lipids. The coatings can coat the entire inside surface of the well or only partial surface. For example, in the case of cylindrical wells, the coating might be present only at the bottom of the well or alternatively also present on the side walls of the cylinders. One distinguishing feature of wells that comprise microtiter plates is that the liquid or other material in each well is kept separated from other wells on the plate. For this reason, different samples such as from different patient's blood or serum can be pipetted into separate wells on the microtiter plate without the different samples mixing together. This is an important property of microtiter plates and allows for example testing of multiple samples in a high-throughput manner. A variety of semi-automated and fully-automated robotic instruments have been developed and are commercially available to process such microtiter plates and are used extensively in the research and diagnostic fields.

Microtiter plates are a preferred substrate type for this invention because they provide an easy to store and handle consumable for both high throughput automation and lower-throughput manual processing in conjunction with the steps shown in FIG. 2A-B. Microtiter plates are essential for processing a large number of samples in parallel. However, they are generally inexpensive enough to be useful in processing even a small number of samples in parallel. Microtiter plates are the industry-standard for a wide range of assays, both high-throughput automated assays and low- to medium-throughput semi-automated or manual assays. A wide range of industry-standard equipment and instrumentation exists for the storage, handling and processing of microtiter plates, including liquid handling robotics, multi-channel pipettors, multi-drop dispensers, plate shakers, plate washers, incubators and automated plate sealers.

However, while microtiter plates offer these important advantages, it is critical that the wells of the microtiter plate also possess several additional properties which are not incorporated into microtiter plates used and/or described in the art, and that would enable the plates to effectively concentrate and/or purify the analytes as in the case of biomarkers from blood, serum, plasma and other biofluids. These critical properties, as they relate to binding agents attached to the plates by a photocleavable linker as used in the PC-PURE process, include but are not limited to: 1) providing sufficient binding capacity in the well for loading of the photocleavable binding agent so that it can bind a significant fraction, ideally 100%, of the analyte from the volume of liquid sample. This feature is particularly important in cases where the concentration of the analyte in the sample is sufficiently high (e.g. IgG in serum) and the collected volume sufficiently large, which would normally saturate the photocleavable binding agent and thus result in the capture of less than the total analyte from the volume of liquid sample collected. Partial capture of the analyte can result in inaccurate measurement of the analyte such as in a quantitative diagnostic assay. 2) The mechanism for concentration of the analyte involves reducing the amount of volume of the buffer (release volume) relative to the volume of the collected sample containing the analyte (sample volume). Thus, the area of contact between the material containing the photocleavable binding agents and the release volume will also be constrained. For example, coating the walls in addition to the bottom of a cylindrical microtiter plate well with a photocleavable binding agent will increase the binding capacity of the well for the analyte, but will prevent reducing the release volume during photocleavage below the height where the well walls are coated, thereby impairing the ability to concentrate the analyte. In order to achieve maximum concentration, it is highly desirable that only the bottom of the well be coated with the medium (substrate) containing the photocleavable binding agents (yet a high density of binding agent must be present in this area). In another configuration of well shape, such as a conical- or V-shaped well, the same considerations hold, whereby it is advantageous to coat only the tip of the conical- or V-shaped well (but again coating must be at high density for maximum concentrating) in order to recover the photocleaved analyte into a minimum volume of fluid. The surface area of contact between liquid in the well and the material which contains the photocleavable binding agent must be minimized in order to allow for optimal concentration of the analyte upon photo-release of the binding agent and hence the analyte into the release volume of liquid. For example, the binding agent may be focused (at high density) only on the bottoms of the wells. Together, these traits would allow for not only purification of the analyte, but also concentration (by photo-releasing in a smaller volume compared to the original sample volume). Conversely, if the photocleavable binding agent were spread/diffuse over the whole surface of the well (sides and bottom), concentrating the analyte would be less effective (due to the need to photo-release in large volumes to recover all of the isolated analyte). Desirable microtiter plate traits can be achieved using the substrate geometries and materials described in detail herein (e.g. microtiter plates with high loading-capacity gels, films or membranes forming or costing only the bottoms of the wells).

Microtiter plate wells with U-, V- or conical-bottoms (with the photocleavable binding agent focused at high density on the well bottom) may facilitate photo-release in very small volumes for the greatest concentrating effect. However, flat-bottom wells coated with a high density of photocleavable binding agent on the well bottom are also effective (see Experimental Examples). Deep-well microtiter plates can facilitate the addition of large initial sample volumes (up to 2 mL for standard deep-well types versus 0.3 mL for normal depth microtiter plates), also increasing the ability to concentrate the analyte.

In contrast, agarose beads can also be used in this invention as the substrate but are less desirable even though they are one of the most widely used resins for affinity isolation (due to their high capacity and hydrophilic/bio-compatible material). Generally, such beads require several time-consuming and poorly automatable steps when used in conjunction with the embodiment illustrated in FIG. 2A-B, including: i) dispensing agarose bead suspensions, which rapidly settle, making this a difficult process to automate and perform reproducibly; ii) vacuum filtration (e.g. in microtiter filter plates) to process the agarose beads for removal of non-captured material in the sample matrix (alternatively, processing the beads by pelleting using centrifugation and removal of the fluid supernatant is prohibitively cumbersome for large sample numbers and high-throughput automation); iii) the need to pre-filter the sample to avoid clogging during this step (pre-centrifugation is insufficient in some cases, especially where the solid debris in the sample are less dense than the liquid component of the sample—as can be the case with serum); and iv) agarose beads (like most beads, microspheres or particles) cannot be frozen or easily dried (e.g. without aggregation), making long-term storage difficult. These factors listed above are also features associated with not just agarose beads, but the use of most beads, microspheres or other particles. In general these factors result in storage problems, long processing times, more expensive automation equipment and decreased accuracy compared to the use of microtiter plates.

Microtiter plates are also to be distinguished from microarrays, whereby microarrays are not suitable for the concentrating and/or purifying of analytes from samples as described in the present invention. Those of skill in the art refer to microarrays. A microarray is a positionally addressable array, such as an array on a solid support, in which the loci of the array (sometimes referred to as probes, features or spots) are at high density. A critical distinguishing feature of a microarray compared to a microtiter plate is that each loci on the array is not isolated from other loci such that a liquid placed on the microarray will contact all loci. Thus, unlike wells in a microtiter plate, loci on a microarray are simultaneously exposed to the same sample. Another important distinguishing feature between microtiter plates and microarrays is that the capture/isolation and then photo-release of analytes, such as biomarkers, for the purpose of concentrating and/or purifying said analytes from a sample can be performed in separate wells of a microtiter plate, thus facilitating processing of multiple samples, but cannot be performed for multiple samples on a single microarray. Importantly, a typical array formed on a surface the size of a standard 96-well microtiter plate (128×86 mm) with 96, 384, or 1,536 loci, is not a microarray [U.S. Patent Application No. 20040241748, Ault-Riche et al.]. Arrays at higher densities such as greater than 2,000, 3,000, 4,000 and more loci per plate (or support) are considered microarrays (whether it be on a support the size of a microtiter plate, or otherwise, for example, commonly the size of a microscope slide at 75×25 mm). Thus, microarrays are high density arrays such that the number of loci per mm² is greater than 0.2 loci/mm², 0.3 loci/mm², 0.35 loci/mm², 0.4 loci/mm² or greater. Any array containing three or more loci in which the loci are at such densities is a microarray.

Whatever the substrate type, materials comprising the substrate may include, but are not limited to, any one of the following or any combination thereof: metals; plastics; polymers; glass; silica; magnetic and paramagnetic materials; cellulose, nitrocellulose (cellulose nitrate), cellulose acetate and other cellulose esters; agarose: dextran; polystyrene, including as cross-linked with divinylbenzene and the like; polypropylene; polycarbonate; polyethyleneglycol (PEG); latex; polyacrylamide; polyvinylidene fluoride (PVDF); polyethersulfone (PES); and the like.

Substrate materials may also be coated with (including by passive adsorption) or chemically modified with various compositions to facilitate immobilization of the binding agent. Said compositions include but are not limited to, succinimidyl esters. N-hydroxysuccinimidyl (NHS) esters, acrylates, biotin, maleimide, iodoacetamide, azide, hydrazides, aldehydes, alkynes, carboxyls, amines, sulfhydryls, avidin, streptavidin, or NeutrAvidin. In one preferred embodiment, substrates are coated with avidin, streptavidin, or NeutrAvidin and are used to immobilize binding agents conjugated to a photocleavable biotin (PC-Biotin) [Olejnik, Sonar et al. (1995) Proceedings of the National Academy of Science (USA) 92; 7590-7594].

Substrates may be comprised of solid (non-porous) materials or porous materials (such as micro-porous, i.e. having micron-scale pores) or a combination thereof. Substrates may be comprised of gels, films or membranes, or any combination thereof, for example, gels, films or membranes which coat or form the bottom of a well of a microtiter plate, as detailed below:

Fabrication of thin film gels: Thin film gel formation can be based on literature reports which have made such gels/films for different purposes, such as tissue engineering, microfluidics and cell culture studies [Gustavsson and Larsson (1999) J Chromatogr A 832; 29-39; Rubina, Dementieva et al. (2003) Biotechniques 34: 1008-14, 1016-20, 1022; Yang, Nam et al (2008) Ultramicroscopy 108: 1384-9; Lee, Arena et al. (2010) Biomacromolecules 11: 3316-24; Strecker, Wumaier et al. (2010) Proteomics 10: 3379-87; Mih, Sharif el al. (2011) PLoS One 6: e19929; Byun, Lee et al. (2013) Lab Chip 13: 886-91; Francisco, Mancino et al. (2013) Biomaterials 34: 7381-8; Kim and Herr (2013) Biomicrofluidics 7: 41501; Francisco, Hwang et al. (2014) Acta Biomater 10: 1102-11]. In one example, a thin (˜60 μm) protein-modified polyacrylamide gel was cast into microtiter plates [Mih, Sharif et al. (2011) PLoS One 6: e19929]. Based on these reports, gel types can include PEG based hydrogels, agarose gels and polyacrylamide gels, including macro-porous gels to allow for rapid macromolecule (e.g. protein) diffusion. Polymerization methods include chemical, photo-polymerization or simple temperature control in the case of agarose. Functional groups can be covalently co-polymerized into the gels for later attachment of streptavidin for example (e.g. to immobilize PC-Biotin conjugated binding agents). Functional groups that can be co-polymerized include but are not limited to bifunctional PEG derivatives commercially available from Creative PEGWorks, such as Acrylate-PEG-Biotin for later attachment of tetrameric streptavidin, Acrylate-PEG-Carboxyl/Amine so that standard carbodiimide (e.g. EDC) and N-hydroxysuccinimide (NHS) ester chemistries can be used for subsequent streptavidin attachment, and Acrylate-PEG-NHS/Maleimide to directly attach to amines or sulfhydryls in the streptavidin. Reactive groups can also be introduced into the gels after polymerization, such by using sulfo-SANPAH, which upon photo-activation introduces a protein-reactive NHS ester into the gel [Mih, Sharif et al. (2011) PLoS One 6: e19929], which can be used to immobilize streptavidin.

Fabrication of thin film porous membranes: Common high binding capacity (high binding density) porous membranes include nitrocellulose and PVDF (typically 0.45 micron sized pores) to which proteins such as streptavidin can be passively adsorbed (bound), e.g. to subsequently immobilize PC-Biotin conjugated binding agents. Alternatively, intermediate agents can be adsorbed to the membranes, such as biotinylated-BSA, followed by attachment of tetrameric streptavidin, avidin or NeutrAvidin for example. Such indirect methods may better preserve the functional binding activity of the streptavidin, avidin or NeutrAvidin for example. Photocleavable chemical linkers may also be directly attached to the membrane and used to directly attach the binding agents. Commercially available microtiter plate options include 96-well Oncyte® Film Plates (Grace Bio-Labs), which use a 12 micron thick porous nitrocellulose coating (on top of a glass well bottom) to provide high capacity. Nitrocellulose or PVDF microtiter filter plates (where the membrane forms the well bottom) are also available from a variety of vendors such as EMD-Millipore (these plates generally do not leak without applied vacuum and therefore can also be processed in a manner similar to standard solid microtiter plates, without filtration; e.g. by removing liquids from the wells by inversion, pipetting or aspiration). Membranes can also be custom cast into a variety of microtiter plates using published procedures for forming these membranes (e.g. [Ahmad, Low et al. (2007) Scripts Materialia 57: 743-746; Flynn, Arndt et al. (2013) Advances in Chemical Science 2: 9-18]). It is worth noting that although generally non-transparent (but translucent, especially when wet), these membranes are thin enough (typically 10-150 microns) that with sufficient light intensity, photocleavage is possible (see Experimental Examples). Nonetheless, these membranes can often be made transparent by refractive index matching, e.g. nitrocellulose in glycerol or oil for example.

Importantly, these 3-dimensional gels, films or membranes can provide a high binding capacity that is located at high density in the bottoms of the microtiter plate wells, to enable biomarker concentration. For example, according to manufacturer specifications, EMD-Millipore plates (MultiScreen_{HTS} HA Filter Plate) with a 150-micron thick cellulose nitrate/acetate membrane-bottom can bind 150 μg of protein per cm², for approximately 40 μg per well (of a 96-well plate).

### Sample Collection Containers for Immobilizing Binding Agents

This embodiment relates to sample collection containers, that are used to collect samples of biological fluids for clinical diagnostic testing or research purposes. This embodiment includes, but is not intended to be limited to, the small plastic cylindrical containers with caps that are used to collect blood samples and in some instances are used to perform testing for the diagnosis of the disease or health status of a patient. A second example is sample collection cups with screw-on lids used to collect urine samples for urinalysis and to provide for leak-free transport and handling.

Most commonly these containers are designed to simply contain the sample, but sometimes they may also contain additives, such as to aid in the preservation of the sample or preservation of the sample in a particular state (e.g. a liquid state). For example, the Becton Dickenson (“BD”) Microtainer™ or Vacutainer™ blood collection containers, and other similar containers, are available in versions that contain EDTA or Sodium Heparin, which are used to prevent or delay the clotting of a blood sample (for example to facilitate the collection of blood plasma). Other tubes are available that do just the opposite, containing clot activator chemicals which speed coagulation and the associated separation of the sample into a solid blood clot and a liquid portion (serum). These types of tubes may also include a neutrally buoyant gel that separates blood cells and clot from the liquid portion of the sample, to aid in providing serum or plasma that can be extracted for later analysis.

Sample collection tubes may also have features to aid in later handling with greater ease, such as pre-printed bar codes or lids that provide a leak-free membrane that can be punctured and re-sealed for withdrawing a portion of a sample contained within. These same features help facilitate automated handling of the tubes, for example handling with an automated laboratory robotic fluid-handling system. Tubes may also have features that aid in the collection of a sample, such as integral capillary tubes for drawing up a blood drop through capillary action; a “scoop” contoured into the lip of the device to aid in sample collection; or pre-prepared with a negative pressure (vacuum) inside to help “pull up” a sample.

Tubes have been conceived that contain nutrient broth or other cell culture medium to accelerate later analysis by allowing fungi, yeast, or other pathogenic organisms which may be present in the sample to grow to facilitate later analysis.

One preferred embodiment of the invention relates to a novel collection container for biological fluids that has in addition to the aforementioned common features, a substrate on the inside (wall) of the container that facilitates the PC-PURE process, that is, to capture/isolate and then photo-release analytes, such as biomarkers, for the purpose of concentrating and/or purifying said analytes from the biological fluids collected in the sample containers. In some cases, the inside wall of the container itself may be the substrate or the substrate may be a coating or a layer on the inside wall of the container. This substrate may be only on a specific portion of the container's inside—such as the bottom of the sample container, or on the bottom and sides or some combination thereof. The substrate contains the directly or indirectly attached binding agent. For example, in one configuration of this invention, the sample collection tube may contain a layer of nitrocellulose (the substrate) that contains anti-IgE antibodies (the binding agent—discussed in more detail later). When exposed to a blood sample, IgE (the analyte) present in the sample will bind to the anti-IgE antibodies during handling and transport of the collection tube. Later, the contents of the sample tube may either be aliquoted for non-IgE related testing or simply washed out, leaving the IgE bound to the substrate of the tube. The IgE bound to the substrate of the tube can either be assessed directly through the addition of detectable (e.g. fluorescent) compounds which bind to the already-bound IgE; or it can be released into a solvent that has been added to the tube, whereby release can for example be caused by energy such as light of a particular wavelength, heat or chemical reaction. When the release is photo-release (i.e. light mediated), this constitutes the PC-PURE process as performed directly in the sample collection tubes. Release into a volume of solvent that is greater than the initial blood sample can be used to dilute the concentration of the analyte in order to facilitate subsequent analysis of the sample. Release of the bound analyte into a volume of solvent that is less than the volume of the original blood sample can be used to increase the concentration of the analyte (i.e. concentrate the analyte). This concentration or dilution can be used to better match the concentration to the analytical method that will be used later. For example, in an assay for IgE which is a relatively low-abundance analyte (biomarker), it may be desirable to concentrate the sample. In the case of an assay for IgG as the analyte, which is a highly abundant biomarker, it may be desirable to dilute the sample. This concentration or dilution is a method which can also simultaneously purify the analyte, by which sample matrix interference may be eliminated or reduced to optimize an assay.

The solvent containing the released material may optionally be aliquoted and analyzed in a different container such as a 96-weIll plate, or it may be analyzed directly within the sample tube. Analysis directly within the sample tube may optionally be facilitated through the use of racks that are commercially available that permit sample tubes to be arranged within the rack in a foot-print that matches the foot-print of a standard 96-well microtiter plate (or other size microtiter plate).

Through these steps that include (1) a biological fluid sample in a container (2) a subset of the components of the fluid sample (analytes) being bound to the wall of the container (the substrate) (3) Aliquoting from the unbound remnant for other tests and/or washing/discarding of the unbound remnant (4) direct analysis or indirect analysis by release of the components (analytes) into a solvent, potentially including concentration, dilution and/or purification of the analyte; greater efficiency in performing the needed analytical tests can be obtained. This increased efficiency will result in decreased labor and lower costs to the healthcare system.

In particular, assays performed today very commonly commence with the aliquoting of a portion of a liquid sample provided into an analysis container, for example a well in a 96-well microtiter plate. In the embodiment disclosed here, the use of the sample collection container for both collection, transport, handling, analyte capture, analyle purifications optional concentration/dilution of the analyte, and analysis through insertion into, for example, a rack that simulates the dimensions of a 96-well microtiter plate would eliminate a time and labor-consuming aliquoting step which is frequently performed manually. Eliminating the aliquoting step also increases the accuracy of an assay by eliminating a step in which volume could be lost, and error could be inserted into the assay step. Furthermore, concentrating or diluting the analyte within the sample collection container as described here provides a convenient way to compute the dilution or concentration factor since the full container containing the biological fluid can be weighed, and then the container containing solvent can be weighed, and the ratio of the weights used to accurately quantify the dilution or concentration factor.

Finally, the embodiment described here can be applied to solid or semi-solid biological samples such as fecal matter or hair by adding a solvent to the sample in a measured fashion and macerating and/or thoroughly mixing to achieve a uniform consistency. Analytes such as biomarkers within the solid or semi-solid would then be homogenously distributed throughout the mixture and captured by the binding agent on the substrate of the container. This could be particularly useful, for example, as an efficient means to perform cancer-biomarker assays on stool samples.

### Binding Agents

U.S. Pat. No. 8,906,700 is hereby incorporated by reference in its entirety. In a preferred embodiment, the binding agents photocleavably attached to the substrate and having a binding affinity for the analyte are selected from the group consisting of antibodies and fragments thereof [e.g. Fab or F(ab′)2]; single chain variable fragment (scFv) antibodies; single domain antibodies (nanobodies); nucleic acid aptamers; lectins and other carbohydrate binding proteins; engineered protein scaffold based binding agents such as commercially available Affibodies®; antigens including wild-type and modified; Protein A, Protein G, and Protein L; as well as engineered fusions of these binding agents. However, the invention is not intended to be limited to any one type of binding agent, as any binding agent, for example based on amino acid or nucleic acid scaffolds, or combinations thereof, may be used. It is to be understood that modifications of the aforementioned binding agents may also be used. For example, modified, truncated, fused or otherwise altered forms of protein A or G that may be used for analyte concentration and/or purification would also fall within the spirit and the scope of the present invention. Protein A or G might be altered by site directed mutagenesis using techniques well known in the art, to produce a protein with altered characteristics which would also function to bind the analyte. It is understood that such altered proteins, or any functionally equivalent proteins would also fall within the scope of the present invention.

The binding agents may be attached to the substrate by a variety of means, such as by direct chemical attachment (e.g. covalent attachment) or indirectly, such as by attaching a small molecule affinity tag (e.g. biotin or digoxigenin) to the binding agent and then attaching to a substrate coated with a cognate ligand to the affinity tag (e.g. avidin, streptavidin or NeutrAvidin ligands for biotin affinity tags, or an anti-digoxigenin antibody ligand for digoxigenin affinity tags). For direct chemical attachment of binding agents to the substrate, a variety of means can be used. Amine or carboxyl functional groups can be used to attach binding agents to substrates by an amide bond, for example using succinimidyl ester chemistry (e.g. attaching amine-containing antibodies to an NHS-activated amine-reactive substrate) or using carbodiimide chemistry (e.g. attaching amine-containing antibodies to carboxyl-terminated substrates following surface activation by EDC). Epoxy, cyanogen bromide or aldehyde-activated substrates may also be used for direct chemical attachment of binding agents to the substrate.

The attachment of the binding agent to the substrate is made reversible by using photocleavable linkers, allowing release of the binding agent by light (so-called photo-release or photocleavage). A variety of photocleavable linkers (PC-Linkers) have been reported, however, photocleavable linkers based on 2-nitrobenzyl or 1-(2-nitrophenyl)-ethyl moieties are preferred [Olejnik, Sonar et al (1995) Proceedings of the National Academy of Science (USA) 92: 7590-7594; Olejnik (1996) Nucleic Acids Research 24: 361-366; Olejnik, Krzymanska-Olejnik et al. (1998) Methods Enzymol 291: 135-54; Olejnik, Krzymanska-Olejnik et al (1998) Nucleic Acids Res 26: 3572-6; Olejnik, Ludemann et al. (1999) Nucleic Acids Res 27; 4626-31]. U.S. Pat. Nos. 5,643,722 and 5,986,076 are hereby incorporated by reference in their entirety.

Contacting the sample with the binding agent photocleavably attached to the substrate is typically achieved by suspending the substrate, in the case where it is beads, microspheres or particles, or simply combining the substrate in other cases, with the liquid samples to be treated. In one preferred embodiment, this includes incubating the combined substrate and liquid sample with agitation for an appropriate time period at an appropriate temperature so as to promote binding of the analyte in the sample to the binding agent. In an alternate embodiment, the contact can be made in the form of a column or filtration device (containing or comprising the substrate) connected to a peristaltic pump, for example to enhance the flow rate of the sample past the substrate. The contact step may be repeated two, three, four or even more than four times to increase binding of the analyte to the photocleavable binding agent on the substrate.

### Photocleavage and Light Sources

Example light sources used to cleave the photocleavable biotin (PC-Biotin) photocleavable linker (PC-Linker) described extensively in the Experimental Examples include but are not limited to: ELC-500 UV Cure Chamber (Fusionet, LLC, Limington, Me.), Blak-Ray Lamp (UVP, Upland, Calif.) and a FireJet™ FJ800 LED Array (Phoseon Technology, Hillsboro Oreg.). While these sources deliver a peak intensity of 365 nm light (desirable since such wavelengths are less damaging to biomolecules compared to shorter wavelengths), usable light sources are not intended to be limited to any one intensity of output, manner of light delivery, or wavelength. Light within the effective photocleavage range of a given PC-Linker may be used. U.S. Pat. Nos. 5,643,722 and 5,986,076 are hereby incorporated by reference in their entirety.

Cleavage, as referred to herein, is by photocleavage or a cleavage event triggered by the application of radiation to the PC-Linker. The radiation applied may comprise one or more wavelengths from the electromagnetic spectrum including x-rays (about 0.1 nm to about 10.0 nm; or about 10¹⁸ to about 10¹⁶ Hz), ultraviolet (UV) rays (about 10.0 nm to about 380 nm; or about 8×10¹⁸ to about 10¹⁶ Hz), visible light (about 380 nm to about 750 nm; or about 8×10¹⁶ to about 4×10¹⁴ Hz), infrared light (about 750 nm to about 0.1 cm; or about 4×10¹⁴ to about 5×10¹¹ Hz), microwaves (about 0.1 cm to about 100 cm; or about 10⁸ to about 5×10¹¹ Hz), and radio waves (about 100 cm to about 10⁴ m; or about 10⁴ to about 10⁸ Hz). Multiple forms of radiation may also be applied simultaneously, in combination or coordinated in a step-wise fashion. Radiation exposure may be constant over a period of seconds, minutes or hours, or varied with pulses at predetermined intervals.

### Reference Agents

U.S. Pat. Nos. 5,643,722 and 5,986,076 are hereby incorporated by reference in their entirety.

In a preferred embodiment, a reference agent is immobilized on the surface of a well of a microtiter plate by a photocleavable (PC) linker (PC-Linker), similar to PC-Linkers described previously to attach binding agents to substrates, and in addition comprises a detectable moiety. A detectable moiety includes, but is not limited to, a chemical group, structure or compound that possesses a specifically identifiable physical property which can be distinguished from the physical properties of other chemicals present in a heterogenous mixture. This includes, but is not limited to, detectable moieties with specific properties that can be distinguished spectroscopically from other molecules such as wavelength dependent light absorption, fluorescence, vibrational, mass to electric charge ratio and other properties normally familiar to those working in the field of molecular spectroscopy.

A detectable moiety also includes those chemical structures that can be identified due to their selective interaction with other molecules, said other molecules referred to here as detection agents, which exhibit an affinity for the detectable moiety. Detection agents for this later group of detectable moieties includes, but is not limited to, antibodies and fragments thereof [e.g. Fab or F(ab′)2]; single chain variable fragment (scFv) antibodies; single domain antibodies (nanobodies); nucleic acid aptamers; lectins and other carbohydrate binding proteins; engineered protein scaffold based binding agents such as commercially available Affibodies® antigens including wild-type and modified; Protein A, Protein G, and Protein L; as well as engineered fusions of these binding agents. The corresponding detectable moieties for these detection agents include but are not limited to binding partners for these defection agents such as biotin, polyhistidine, digoxigenin and carbohydrates, as well as proteins/peptides and nucleic acid based molecules. For example, a detectable moiety (e.g. digoxigenin) can be detected due to its interaction with the aforementioned detection agents which are part of a solid-phase ELISA assay. Note that the detectable moiety and detection agent are interchangeable. One example is an antibody as the detectable moiety which exhibits a high affinity for its cognate antigen or hapten such as digoxigenin. In this case, the detection of the antibody is based on, for example, interaction with the cognate antigen or hapten which can be part of (e.g. immobilized on the surface of) a solid-phase ELISA.

Typically, the photocleavable reference agents are attached using the same methods and compositions as described earlier for binding agents. However, unlike binding agents, reference agents are chosen to possess the property that they contain a detectable moiety which can be quickly and accurately detected after the reference agent is photocleavably detached from the substrate. In addition, unlike ordinary binding agents, in some cases they are chosen so they do not bind analytes or other compounds present in the sample which contacts the well of the microtiter plate.

In one preferred embodiment, reference agents are photocleavably attached in one or more wells of the microtiter plate to the same substrate which photocleavable binding agents are attached. In this preferred embodiment, the PC-Linkers which attach the photocleavable reference agent and the photocleavable binding agent have identical or very similar properties including similar chemical structures and response to light. Both the photocleavable reference agent and the photocleavable binding agent are photocleaved simultaneously with the same light source as used for the binding agents.

In one embodiment of the invention the photocleavable reference agent consists of a bioreactive agent comprising a detectable moiety bonded to a photoreactive moiety wherein the photoreactive moiety contains at least one group capable of covalently bonding to a substrate located on the inside surface of the well of the microtiter plate to form a conjugate. The resulting conjugate can be selectively cleaved to release said detectable moiety or, alternatively, to release any chemical group or agent of the conjugate which can serve as a detectable moiety.

Detectable moieties include, but are not limited to, a chemical group, structure or compound that possesses a specifically identifiable physical property which can be distinguished from the physical properties of other chemicals present in a heterogeneous mixture. Fluorescence, phosphorescence and luminescence including electroluminescence, chemiluminescence and bioluminescence are all detectable physical properties not found in most substances, but known to occur or to be inducible in others. For example, reactive derivatives of dansyl, coumarins, rhodamine and fluorescein are all inherently fluorescent when excited with light of a specific wavelength and can be specifically bound or attached to other substances. Coumarin has a high fluorescent quantum yield, higher than even a dansyl moiety; and facilitates detection where very low levels of detectable moiety are being sought. Additional examples include chemical groups and compounds with distinctive vibrational spectra which serve as fingerprints to identify the chemical group or compound. Vibrational spectra can be detected using a variety of physical methods including infrared absorption and Raman spectroscopy. In many cases the chemical groups have electronic transitions which can be used to resonance enhance the Raman spectrum many orders of magnitude. It may also be useful to combine different detectable moieties to facilitate detection.

A reference agent can be used for a number of purposes including as a calibrant, quality control agent and photo-exposure agent, during the manufacture, transportation and storage of the microtiter plates as well as during the PC-PURE process and for downstream quantification of analytes. For example, in one preferred embodiment both the reference agent and binding agent are photocleavably attached to a substrate that is in or part of a microtiter plate well through similar or identical PC-Linkers. A biological sample containing analytes such as biomarkers is introduced into the well. Subsequent to the capture of the biomarker by the binding agent, the substrate is washed with a controlled buffer solution and then the well illuminated with the appropriate wavelength and intensity of light so that both the detectable moiety of the reference agent and the captured biomarker bound to the binding agent are simultaneously released into a solution of known volume and composition. The measurement of the amount of photo-released detectable moiety is then used as a means to detect and correct for effects which could lead to in accuracies in measurement of the biomarker analytes.

Downstream Detection, Measurement and/or Quantification of Analyte

Finally, following analyte concentration and/or purification from the sample, the analyte (e.g. biomarker), in a preferred embodiment, is subjected to immunoassay for detection, measurement and/or quantification (it is to be understood however that other methods of measurement, such as mass spectrometry assays, can also be used). Immunoassays can be of a variety of formats, such as homogenous (no-wash) assays including proximity assays based on surface plasmon resonance (SPR), fluorescence resonance energy transfer (FRET), time-resolved fluorescence resonance energy transfer (TR-FRET) or bioluminescence resonance energy transfer (BRET). Alternatively, in a preferred embodiment, heterogeneous assays are used (solid-phase wash-based assays). Such assays include but are not limited to ELISA (enzyme-linked immunosorbent assay), RIA (radioimmunoassay), FEIA (fluorescence enzyme immunoassay), Western blot, dot blot, and lateral flow formats as well as microarray, microsphere (bead) and microfluidics based formats. Immunoassays may be of the sandwich type (e.g. capture antibody immobilized on assay surface which binds the analyte which is also then bound by a detection antibody), immobilized-antigen type (e.g. antigen on assay surface for binding of an antibody/immunoglobulin analyte, which is then detected) or competitive inhibition type (e.g. analyte from sample competes with an analogous but labeled analyte for binding to the assay surface), for example. In a preferred embodiment, the detection, measurement and/or quantification assay is a multiplex assay, such as a microarray or microsphere-based multiplex assay or immunoassay.

Using Binding Agents and Reference Agents Together in the PC-PURE Process and Subsequent Detection, Measurement and/or Quantification in a Subsequent Assay

In one embodiment, a reference agent is attached to a substrate by a photocleavable linker, and a binding agent, which binds an analyte from a sample, is attached to the same substrate also by a photocleavable linker. Said substrate is used for the PC-PURE process to concentrate and/or purify said analyte from said sample using said binding agent attached to said substrate by a photocleavable linker, whereby said reference agent is also photo-released simultaneously along with said binding agent and any bound analyte during said PC-PURE process. Furthermore, said reference agent is configured such that it is detectable in a subsequent assay, an immunoassay for example, by the same mechanism by which said analyte is detected in said subsequent assay. Yet, said reference agent is also configured such that it does not interact with said binding agent, which could otherwise confound the measurement and/or detection of said reference agent in said subsequent assay and/or interfere with the binding function of said binding agent for said analyte. For example, in the case where said analyte is IgE from a serum sample (see Experimental Examples 3-5 and 7-9), said reference agent can be a digoxigenin-labeled non-immune IgE having no specific antigen reactivity (see Experimental Example 1), which has been further conjugated to photocleavable biotin to facilitate attachment to said substrate. Said binding agent is an anti-IgE monoclonal antibody in this embodiment, also conjugated to a photocleavable biotin for substrate attachment, whereby said binding agent binds said IgE analyte from said serum sample. To avoid interaction of said binding agent with said reference agent, said binding agent may be an anti-IgE monoclonal antibody which interacts selectively with the Fc region of said IgE analyte from said sample and said reference agent may be an F(ab) (Fab) or F(ab′)2 fragment of IgE, lacking an Fc region and thus unable to interact with said binding agent. Said subsequent assay can for example be a multiplex microsphere-based immunoassay. Whereby said reference agent is captured on a particular coded microsphere which is coated with an anti-digoxigenin antibody (see Experimental Example 1) and said IgE analyte binds to a different set of coded microspheres each coated with different allergens (antigens), used to bind the allergen-specific IgE fraction of said IgE analyte. In said multiplex immunoassay, both said reference agent and said analyte may then be detected on the respective microspheres using the same anti-IgE antibody, but a different antibody from said binding agent, configured to bind IgE outside the Fc region, within the F(ab) (Fab) or F(ab′)2 regions for example, and either detectably labeled directly (e.g. phycoerythrin; see Experimental Examples 1,3-5 and 7-9) or detectable with a secondary detection agent.

### EXPERIMENTAL

### Example 1

Photocleavable Antibodies (PC-Antibodies) on Beads (PC-Beads) for PC-PURE Processing of Analytes (IgE as Example); Photo-Releasing the Analyte with the Purification Surface and Assay Surface Together for Greater Efficiency

### Materials

(1-(3-dimethylaminopropyl)-3-ethyl-carbodiimide HCl), Sulfo-NHS (N-Hydroxysulfosuccinimide) No Weigh Format, MES (2-(N-Morpholino)ethanesulfonic Acid), hydroxylamine, 3-Amino-3-Deoxydigoxigenin Hemisuccinamide Succinimidyl Ester and 96-well microtiter MagMAX™ Express Reaction Plates were purchased from Thermo Scientific (Waltham, Mass.), Purified human IgE, mouse monoclonal anti-[human IgE] antibody [Clone BE5] phycoerythrin (PE) labeled, the IgG Mouse ELJSA Kit and the Immunoglobulin IgE Human ELISA Kit were from Abcam (Cambridge, Mass.), Mouse monoclonal anti-[human IgE] antibodies (Clones E411 and 4F4cc) were from HyTest (Turku, Finland). Lightning-Link® R-Phycoerythrin (RPE) labeling kits were from Innova Biosciences (Cambridge, UK), PD MidiTrap G-25 Columns, PD SpinTrap G-25 Columns and Streptavidin Sepharose High Performance 34 μm Beads were from GE Healthcare Life Sciences (Pittsburgh, Pa.). 96-well microtiter filter plates (AcroPrep™ Advance Plates with 3.0 μm Glass Fiber pre-filter and 1.2 μm Supor membrane) were from Pall Scientific (Port Washington, N.Y.), 400 μL capacity Ultrafree®-MC Micro-Centrifuge Filter Units, Pore Size 0.45 μm Durapore® PVDF membrane were from EMD Millipore (Billerica, Mass.). Carboxyl-terminated MagPlex® magnetic microspheres were from Luminex® (Austin, Tex.). A mouse monoclonal anti-Digoxigenin antibody (Clone 1.71.256) and the purified natural allergen component protein lactalbumin (Bos d 4) were purchased from Sigma-Aldrich (St. Louis, Mo.). All other allergen component proteins were purchased from Indoor Biotechnologies (Charlottesville, Va.). Whole food extracts were from Allergy Laboratories, Inc. (Oklahoma City, Okla.) and from the Research Department at Greer Allergy Immunotherapy (Lenoir, N.C.). The PC-Biotin-NHS labeling reagent was from AmberGen (Watertown, Mass.) [Olejnik, Sonar et al. (1995) Proceedings of the National Academy of Science (USA) 92: 7590-7594].

### Photocleavable-Biotin (PC-Biotin) Labeling of an Anti-IgE Antibody: PC-Antibody

Mouse anti-human IgE antibody as supplied (Clone E411) was supplemented to 100 mM sodium bicarbonate from a 1M stock. 15 molar equivalents of PC-Biotin-NHS labeling reagent were immediately added (from a 50 mM stock in anhydrous DMF) to the anti-IgE antibody. The reaction was carried out for 30 min with gentle mixing, protected from light. The reaction was then stopped by adding 1/9th volume of NHS Quench Buffer (200 mM glycine in 200 mM sodium bicarbonate and 200 mM NaCl) and subsequently mixing for 15 mln. To remove unreacted PC-Biotin, the reaction mix was desalted on a PD MidiTrap G-25 Column, performed according to the manufacturer's instructions (equilibration and elution in TBS; 50 mM Tris, pH 7.5, 200 mM NaCl). Following desalting, the final product corresponding to the PC-Biotin labeled anti-IgE antibody (PC-Antibody) was aliquoted and stored at −70°C. The antibody was quantified using a commercial IgG Mouse ELISA Kit.

### Attaching PC-Antibody to Agarose Beads; PC-Beads

120 μL packed volume of Streptavidin Sepharose High Performance 34 μm Beads was washed 3× 1,500 μL briefly in TBS-T by sequential mixing, pelleting the beads briefly in a micro-centrifuge and removing the supernatant. All washes were performed in this manner unless otherwise indicated. The 120 μL bead pellet was then re-suspended in 1,200 μL of PC-Antibody solution (In TBS-T), yielding a ratio of 5 μg of total PC-Antibody per each 1 μL of packed bead pellet volume. Following gentle mixing for 30 min at room temperature, the beads were washed 4× 1,500 μL in TBS-T. Finally, the beads were re-suspended in 480 μL TBS-T to yield a 20% (v/v) bead suspension. Beads (hereafter referred to as PC-Beads) were stored at +4° C. protected from light.

Preparation of Digoxigenin Labeled IgE (Dig-IgE) to Measure Capture and Photo-Release from PC-Beads

Purified human IgE was digoxigenin labeled to create the Dig-IgE as follows: The IgE as supplied was supplemented to 100 mM sodium bicarbonate from a 1M stock. A 10-fold molar excess of 3-Amino-3-Deoxydigoxigenin Hemisuccinamide Succinimidyl Ester labeling reagent was added from a 1 mM stock in DMSO. The reaction was carried out for 30 min with gentle mixing, protected from light. The reaction was then quenched by adding 1/9th volume of 1 M glycine and subsequently mixing for 15 min. To avoid losses in the subsequent desalting column, a BSA carrier was then added from a 10% (w/v) stock to yield a final 0.05% (w/v). To remove unreacted labeling reagent, the reaction mix was then desalted on PD SpinTrap G-25 columns. The PD SpinTrap G-25 columns were performed according to the manufacturer's instructions (equilibration in 300 μL of TBS). Following desalting, the final product corresponding to the digoxigenin labeled human IgE (Dig-IgE) was supplemented with 1/9th volume of 10× TBS before aliquoting and storing at −70° C. The yield of Dig-IgE was quantified using the commercial Immunoglobulin IgE Human ELISA Kit. The Dig-IgE was the analyte in this Example as detailed below.

### Anti-Digoxigenin Antibody Attachment to MagPlex® Microspheres

250,000 carboxyl-modifled MagPlex® microspheres were briefly washed in a micro-centrifuge tube 3× 800 μL with MES Buffer (0.1 M MES, pH 4.7, 0.9 % NaCl) using a magnetic separator, 200 μL of Sulfo-NHS Buffer (1 mg/mL in MES Buffer) followed by 200 μL of EDC Buffer (1 mg/mL in MES Buffer) was added to the washed microsphere pellet. Following incubation with mixing for 1 h the microspheres were then washed 3× 800 μL briefly with MES Buffer. The antibody coupling reaction immediately followed, in which 250 μL of 1 mg/mL anti-Digoxigenin antibody in PBS (48 mM sodium phosphate, pH 7.5, 100 mM NaCl) was added to the microspheres and incubated with mixing for 1 h. The microspheres were then briefly washed 2× 800 μL with Microsphere Quench Buffer (10 mM hydroxylamine in PBS-T; PBS-T contains 0.2% [v/v] Tween) before discarding the wash and incubating with an additional 400 μL of Microsphere Quench Buffer for 30 min with mixing. Microspheres were then washed briefly 1×800 μL with PBS-1M NaCl, 1× 400 μl for 30 min with PBS-1M NaCl (with mixing) and then 2× 800 μL briefly with TBS-T (50 mM Tris, pH 7.5, 200 mM NaCl, 0.05% [v/v] Tween-20). Microspheres were stored, protected from light, in TBS-T at +4° C.

PC-PURE on IgE using PC-Beads Followed by Analyte Measurement on a Multiplex Microsphere-Based Immunoassay Platform

Processing of PC-Beads for IgE purification was done in 96-well microtiter filter plates using a vacuum manifold, unless otherwise specified (processing of PC-Beads could also be performed in micro-centrifuge filter units; see Materials). 5 μL bead pellet volume of PC-Beads (1-5 μL bead pellet volume was typically used) per well was washed briefly 4× 200 μL with TBS-T followed by the addition of 100 or 200 μL of IgE containing sample, in this case a 12,500 pg/mL Dig-IgE sample in 5% BSA (w/v), TBS-T. PC-Beads and sample were mixed together for 1 h to allow capture of the Dig-IgE from the sample onto the PC-Beads. PC-Beads were washed 4× 200 μL briefly followed by 3× 200 μL for 10 mm each (with mixing) with TBS-T (10 min washes were typically employed for complex bio-samples such as serum or plasma, and were omitted for simpler sample matrices). Photo-release of the [PC-Antibody]-[Dig-IgE] complexes from the PC-Beads was performed and then followed by incubation of the supernatant with the anti-Digoxigenin antibody-coated microsphere assay surface (Sequential Method), or for greater efficiency, photo-release was performed in the presence of the anti-Digoxigenin antibody-coated microspheres (Combined Method). In either case, photo-release was achieved by illuminating with 365 nm light for 60 min using a Blak-Ray Lamp Model XX-15 (UVP), or 20 min using an ELC-500 UV Cure Chamber (Fusionet, LLC) or 100 to 200 s using a FireJet™ FJ800 LED Array (Phoseon). Typical distance from the light source was 5-10 cm. For the Sequential Method, photo-release was performed in BSA Block (1% BSA [w/v] in TBS-T), the fluid supernatant collected (no PC-Beads) and the supernatant combined with the anti-Digoxigenin antibody-coated microsphere assay surface (2,500 microspheres/sample; note that a constant final volume was maintained compared to the original sample volume input to the PC-Beads). For the Combined Method, anti-Digoxigenin antibody-coated MagPlex® microspheres (2,500 microspheres/sample) were suspended in BSA Block and added to the washed PC-Bead pellets followed by photo-release (again, a constant final volume was maintained compared to the original sample volume input to the PC-Beads). Photo-release was also performed in some cases in plain TBS-T without BSA. In any case, post-photo-release mixing was next performed for 30 min to allow the photo-released [PC-Antibody]-[Dig-IgE] complexes to be re-captured onto the MagPlex® anti-Digoxigenin antibody-coated microspheres.

The immunoassay was completed using the MagMAX™ magnetic particle processing robot (Thermo Scientific). MagPlex® microspheres were transferred into a deep-well microtiter plate and washed briefly 3× 900 μL with TBS-T. Microspheres were then probed for 30 min with mixing using 100 μL/well of 1 μg/mL phycoerythrin-labeled monoclonal mouse anti-[human IgE] antibody in BSA Block. Microspheres were then washed 3× 900 μL with TBS-T and re-suspended in 100 μL of TBS-T for readout in a Luminex® MagPix® instrument. The immunoassay was also performed manually in some cases, using a magnetic separator slab (Luminex®) which attaches to the bottom of the microtiter plate, immobilizing the magnetic microspheres and allowing removal or decanting of the fluid from the wells of the microtiter plate while retaining the microspheres. In this case, washes were 4× 250 μL with TBS-T.

### Results

The first step in this Example of using Dig-IgE as the model analyte (biomarker), was to prepare an anti-IgE photocleavable antibody (PC-Antibody) which was suitable for isolating total IgE (total Dig-IgE in this case) prior to its input into a solid-phase immunoassay for quantification. For this purpose, a mouse monoclonal anti-IgE antibody, which binds the Fc region of human IgE, was labeled with photocleavable biotin (PC-Biotin) to create the PC-Antibody. The PC-Antibody was then loaded at 25 μg per 5 μL of streptavidin agarose bead pellet volume to create the PC-Beads (as detailed in later Examples, 5 μL PC-Beads was used for each patient serum sample). Binding assays shown in FIG. 3a indicate that 99.7% of the added PC-Antibody was bound by the streptavidin agarose beads (to form the PC-Beads). Next, to measure the IgE binding and photo-release capability of the PC-Beads, a digoxigenin labeled human IgE tracer was prepared (Dig-IgE). The digoxigenin moiety conjugated to the IgE provided a convenient affinity tag to allow quantification of the Dig-IgE using a Luminex® microsphere-based sandwich immunoassay, where an anti-digoxigenin antibody-coated microsphere captures the Dig-IgE which is then detected using a fluorescently labeled anti-IgE detection antibody (the detection antibody, which is the same detection antibody used for the serum sIgE assays detailed in later Examples, binds a different epitope on the IgE than the PC-Antibody). Dig-IgE at 12.5 ng/mL (˜5 kIU_{A}/L) was captured by the PC-Beads (5 μL bead pellet), the PC-Beads then washed and photo-release performed (constant volumes were maintained at every step—thus the Dig-IgE was only isolated and purified but not concentrated in this Example). The amount of Dig-IgE was quantified at each step in the process using the aforementioned sandwich immunoassay, which employed interpolation from a Dig-IgE standard curve. Analyzed were the “Input” (solution prior to adding to PC-Beads), “Depleted” fraction (solution after treatment with PC-Beads) and the “Photo-Released” fraction (solution after UV treatment of PC-Beads). The PC-Bead washes contain negligible amounts (shown in later Examples) and therefore were not analyzed in this Example. In FIG. 3b, with the “Sequential” Method, photo-release was followed by applying the supernatant to the immunoassay microspheres, whereas in the “Combined” Method, photo-release was performed with the PC-Beads and microspheres together. Results in FIG. 3b show that the PC-Beads depleted 100% of the added Dig-IgE and 35% was recovered in the Photo-Released fraction with the Sequential Method. An improvement, 59% recovery in the Photo-Released fraction, was obtained with the Combined Method. Importantly, in addition to the increase in recovery, the Combined Method eliminates steps (transfer of photo-released supernatant from PC-Beads to the microsphere assay surface), simplifying the procedure and making it more amenable to automation.

The apparent lack of 100% photo-release recovery may actually be a result of the lower-efficiency binding to the Luminex® microsphere surface of the [Dig-IgE]-[PC-Antibody] complexes (in Photo-Released fraction) versus the Dig-IgE alone (in Input solution and Depleted fraction), thereby underestimating the amount in the Photo-Released fraction. The Dig-IgE bound PC-Antibody may also partially sterically hinder the binding of the detection antibody in the sandwich immunoassay. It is also possible that a percent of the [Dig-IgE]-[PC-Antibody] complexes remain tightly and non-specifically bound to the PC-Bead surface, and cannot be photo-released.

### Example 2

Binding Capacity of Photocleavable Antibody Beads (PC-Beads) used for PC-PURE: IgE Analyte (Biomarker) as an Example

While results in Example 1 demonstrate the basic function of the PC-Beads to capture and photo-release an analyte (biomarker), they do not estimate the maximum binding capacity of the PC-Beads. The PC-Beads should ideally be able to bind the foil complement of analyte (biomarker) in a sample (e.g. patient blood sample). In the example of IgE as the analyte (applicable to allergy testing, such as measuring allergen-specific IgE), even the most extreme cases must be considered, such as atopy where total IgE levels in a patient's blood are significantly elevated. Upper limits of normal are between approximately 150 and 300 kIU_{A}/L [Laurent, Noirot et al. (1985) Ann Med Interne (Paris) 136: 419-22; Carosso, Bugiani et al. (2007) Int Arch Allergy Immunol 142: 230-8]. In one study on individuals with atopic dermatitis, values ranged as high as 12,000 kIU_{A}/L [Ott, Stanzel et al. (2009) Acta Derm Venereol 89: 257-61]. To estimate the PC-Bead binding capacity, a simple depletion assay was performed as in Example 1 (also see Example 1 for Materials), whereby 5 μL pellet volume of PC-Beads was used to deplete various known amounts of human IgE spiked into a 5% BSA/TBS-T solution (native IgE in this case, not Dig-IgE). Similar to the procedure described Example 1, the Input solution and Depleted fraction were collected and IgE was quantified (in this case using a standard colorimetric human IgE ELISA). Washes from the PC-Beads after IgE capture were also quantified and found to contain less than 3% (in all washes combined) of the total IgE added. The “Un-Captured” IgE amount was considered as the sum of IgE in the Depleted fraction and all washes. Results shown in FIG. 4 indicate that the PC-Beads captured 99%, 94% and 74% of the IgE from 5 μg/mL (˜2,000 kIU_{A}/L), 50 μg/mL (˜20,000 kIU_{A}/L) and 250 μg/mL (˜100,000 kIU_{A}/L) solutions, respectively (at 100 μL sample volume, this was 0.5, 5 and 19 μg of IgE captured; note 2.4 μg=1 kIU_{A}). This demonstrates that sufficient capacity exists to bind the full complement of patient total IgE, even in the most severe cases. Furthermore, the PC-Antibody was highly efficient, with 5 μL PC-Beads containing 25 μg of anti-IgE PC-Antibody (see Example 1) able to capture up to 19 μg of total IgE.

### Example 3

PC-PURE for Eliminating the Matrix Effect with In Vitro Allergy Assays

### Allergen Preparation and Attachment to Microspheres

See Example 1 for Materials. All crode allergen extracts except peanut were prepared at 5 mg/mL in PBS with 5 mM EDTA. Crude peanut extract was prepared at 5 mg/mL in 200 mM carbonate-bicarbonate buffer, pH 9.4, with 5 mM EDTA. The purified natural lactalbumin (Bos d 4) component protein was prepared at 5 mg/mL in PBS with 5 mM EDTA. All other allergen component proteins were prepared at 0.5 mg/mL in PBS with 5 mM EDTA. All allergen preparations were clarified by 1 min micro-centrifugation at 14,000 rpm. Attachment of the allergens to the Luminex® MagPlex® microspheres was done as in Example 1 except that the prepared allergen solutions were used instead of the anti-Digoxigenin antibody solution.

PC-PURE of IgE From Patient Serum/Plasma using PC-Beads Followed by Measurement of Allergen-Specific IgE on a Multiplex Microsphere-Based Immunoassay Platform

Performed as in Example 1 except that endogenous patient IgE in serum/plasma samples was subjected to PC-PURE instead of Dig-IgE in buffered solutions; and the subsequent multiplex Luminex®-based immunoassay used the aforementioned allergen-coated microspheres (microspheres of various species are pooled for the multiplex assay) in order to measure allergen-specific IgE, instead of using anti-Digoxigenin microspheres to measure Dig-IgE. The “Combined Method” during the photo-release step was used as detailed in Example 1. Furthermore, as a comparison, crude (not processed by PC-PURE) serum/plasma was analyzed in the microsphere-based immunoassay to measure allergen-specific IgE (but directly from crude samples). This was performed in the same manner as above except PC-PURE was omitted and crude serum/plasma was input directly into the microsphere-based immunoassay, instead of IgE purified from patient samples using PC-PURE.

### Results

In order to test the ability of PC-Antibody based IgE purification (PC-PURE) to eliminate the matrix effect, it was used as the “front-end” for a multiplex blood-based allergy immunoassay, termed the AllerBead assay, which is based on the Luminex® coded microsphere platform. The overall combined process was illustrated by way of example in FIG. 2A-B, and the embodiment in this Example consists of the following steps: 1) The blood sample was collected and converted to serum or plasma; 2) Total IgE from the serum or plasma was then captured by an anti-IgE photocleavable antibody (PC-Antibody) immobilized on agarose beads (PC-Beads); 3) The PC-Beads were then washed in microtiter filter plates with a controlled buffer solution to remove interfering sample matrix constituents; 4) The [PC-Antibody]-[IgE] complexes were then gently photo-released in minutes from the PC-Beads using 365 nm light; 5) The purified photo-released complexes were re-captured on the multiplex assay surface (Luminex® microspheres in this Example) which were coated with specific allergen extracts or allergen component proteins to bind allergen-specific IgE (sIgE); 6) The assay was read (Luminex® MagPix® instrument in this Example) for detection and quantification. Note that sIgE detection on the Luminex® microsphere was through a separate anti-IgE antibody (labeled with phycoerythrin [PE]), which binds a different epitope on the IgE than the PC-Antibody (see also Example 1).

The multiplex AllerBead assay typically used whole food extracts (one extract coated onto a particular coded microsphere), since these extracts provide clinically useful information and are used commonly for non-multiplex in vitro allergy testing [Lieberman and Sicherer (2011) Curr Allergy Asthma Rep 11: 58-64; Sampson, Aceves et al. (2014) J Allergy Clin Immunol 134: 1016-25 e43]. The whole food extracts typically used for the multiplex AllerBead assay represented the eight most common food allergens (milk, wheat, soy, peanut, tree nut [cashew], egg [white], fin fish [cod] and shellfish [shrimp]) which account for >90% of all pediatric food allergies [Branum and Lukacs (2008) National Center for Health Statistics (NCHS) Data Brief: 1-8]. In order to achieve higher analytical sensitivity, two additional allergens besides the whole food extracts were sometimes utilized since these allergens exist at low relative abundance in the whole food extracts. These component proteins were Ara h 8 for peanut and lactalbumin (Bos d 4) for milk. Note that ImmunoCAP® in some cases has been reported to supplement their allergen extracts with component proteins for higher analytical sensitivity [Sicherer, Dhillon et al. (2008) J Allergy Clin Immunol 122: 413-4, 414 e2]. In all, the 8 whole food extracts and two component proteins resulted in a 10-plex AllerBead assay used in much of the work described herein (in some cases, a subset of these allergens was used).

In this Example, linearity of serum dilution was tested in the AllerBead assay with and without the PC-Antibody based IgE pre-purification approach (PC-PURE). The same PE-labeled anti-IgE detection antibody was used for both AllerBead assay formats and at the same concentration (as noted earlier, this antibody binds a different epitope on the IgE than the PC-Antibody; while the PC-Antibody is not labeled for detection). FIG. 5 shows data from a representative serum dilution series from a patient positive for milk sIgE but negative for soy (as determined using the gold-standard, FDA-cleared, non-multiplex immunoCAP® assay). Without PC-PURE, AllerBead shows apparent saturation for milk sIgE at ˜10% up to 100% crude serum (100 μL input to AllerBead), as evidenced by the plateaued signals, with rapidly decreasing signal below 10% crude serum. However, this does not actually reflect a real saturation of the sIgE binding. The Luminex® microspheres are actually saturated by interfering components from the serum (bound to the microspheres but not detected) and not saturated with the sIgE analyte (which is detected). This is evidenced by the PC-PURE approach which extends the linear range of milk sIgE detection to a much higher signal intensity, approximately 3-fold above the crude serum plateau in this case (200 μL input serum to PC-Beads and 100 μL photo-release volume for analysis in AllerBead assay, to compensate for the roughly 50% losses upon IgE purification as measured earlier in Example 1; regardless of whether the IgE was concentrated or not, that PC-PURE yields linear response extending approximately 3-fold above the plateaued signals of the crude serum demonstrates a removal of the matrix effect). For the full serum dilution series, R² of the linear regression (for milk) was >0.98 for AllerBead with PC-PURE, compared to <0.2 for AllerBead without PC-PURE (crude serum). Critically, the matrix effect is not simply eliminated by diluting the crude serum, since the plateaued signal quickly drops below ˜10% serum. This may be attributed to the feet that simply diluting the serum does not change the ratio of interfering agents (matrix constituents) to the target agent (sIgE), while PC-PURE does. Finally, specificity was maintained with the PC-PURE approach, as evidenced by soy which shows essentially no signal in AllerBead (this patient serum was negative for soy sIgE as determined by the gold-standard ImmunoCAP® test).

### Example 4

Large-Scale Studies using PC-PURE in Multiplex Allergen-Specific IgE Immunoassays (AllerBead Assay): Comparison to Non-Multiplex Gold Standard ImmunoCAP® Assay

See Example 1 for Materials. PC-PURE and the AllerBead assay were performed as in Example 3 with the following exceptions: In this Example, a much, larger assessment of the ability of PC-PURE to improve the AllerBead assay was performed. A total of 205 serum samples obtained in collaboration with Boston Children's Hospital (BCH) were used for this work. The AllerBead 10-plex assay (described in Example 3) was used to quantitatively measure sIgE concentration from the crude serum and the same assay applied to IgE pre-purified from serum (“PC-PURE”). In addition, results were compared to the gold-standard PDA-cleared ImmunoCAP® assay (performed commercially on crude serum by the Phadia Immunology Reference Laboratory [PiRL]). Note that the ImmunoCAP® assay is non-multiplex and was performed for each sample for all 8 whole food extracts. For the AllerBead assay without PC-PURE, 100 μL of serum was used as the input sample volume. For PC-PURE, to ensure in this case that any benefits were strictly from removal of the matrix effects, IgE was isolated from 100 μL of serum and the photo-release volume was also 100 μL, which was input into the subsequent AllerBead assay (thus the IgE was only purified and not concentrated in this Example).

### Results

Key results for the AllerBead assay with and without PC-PURE are summarized in FIG. 6A-C, including comparisons to ImmunoCAP®. AllerBead signal-to-noise (FIG. 6a) was markedly improved using PC-PURE, by up to 18-fold on average for peanut. The smallest increase was 2-fold for cod. Correlation of AllerBead with ImmunoCAP® was determined by Pearson analyses (FIG. 6b), Pearson's r value for AllerBead using PC-PURE averaged 0.90 across the different foods, with all foods ≥0.90 except milk (0.79) and soy (0.86), Pearson hypothesis testing (H₀: r≤0.5) yielded p-values <0.0001 for all foods. In contrast, AllerBead performed without PC-PURE yielded poor ImmunoCAP®-correlation, with an average Pearson's r of 0.62, falling as low as 0.38 for peanut. Furthermore, p-values were >0.25 for four foods. To calculate sensitivity (percent of ImmunoCAP®-positive patients detected by the AllerBead assays), a scoring cutoff for each food was set at 3 standard deviations above the mean AllerBead result for the ImmunoCAP®-negatives (analytical negatives are defined as <0.10 kIU_{A}/L by the ImmunoCAP® assay). AllerBead sensitivity (FIG. 6c) was defined as the percent of mmunoCAP®-positives detected in the range of the maximum measurable by ImmunoCAP® (100 kIU_{A}/L) down to the cutoffs for 95% negative predictive value (NPV) for determining clinical allergy [Sampson and Ho (1997) J Allergy Clin Immunol 100: 444-51; Sampson (2001) J Allergy Clin Immunol 107: 891-6; Perry, Matsui et al. (2004) J Allergy Clin Immunol 114: 144-9], since this is the clinically useful range (see Table 1 for details on the NPV cutoffs, which ranged from 0.35 kIU_{A}/L to 5 kIU_{A}/L depending on the food). Sensitivity of AllerBead with PC-PURE, in this range, averaged 96% for all foods (all ≥94% except soy at 88%). Conversely, sensitivity of AllerBead without PC-PURE averaged only 59%, dropping as low as 23% for wheat.

FIG. 7 shows a sample regression analysis between AllerBead and ImmunoCAP® for cashew, with and without PC-PURE for the AllerBead assay. In the case of PC-PURE, a Pearson's r value of 0.94 (slope of 0.81) was obtained, indicating an excellent correlation of AllerBead with ImmunoCAP®. In contrast, the regression analysis of AllerBead without PC-PURE yields a Pearson's r value of only 0.53 (slope 0.10), indicating an poor correlation of AllerBead with ImmunoCAP®.

Overall, the improvements in AllerBead provided by PC-PURE were achieved despite the fact that the patient IgE was only purified but not concentrated in this Example. The improved signal-to-noise ratio was reflected in the improved AllerBead sensitivity for detecting ImmunoCAP®-positives (FIG. 6c; average 96% for all foods with PC-PURE and 59% without). Thus, PC-PURE eliminates signal suppression in the multiplex immunoassay which is caused by the serum matrix. At least part of this is expected to be the result of eliminating the competitive binding of non-IgE allergen-specific immunoglobulins (e.g. IgG and IgA) [Hofman (1995) Rocz Akad Med Bialymst 40: 468-73; Visco, Dolecek et al. (1996) J Immunol 157: 956-62; Kadooka, Idota et al. (2000) Int Arch Allergy Immunol 122: 264-9; Jarvinen, Chatchatee et al. (2001) Int Arch Allergy Immunol 126: 111-8; Shreffler, Lencer et al. (2005) J Allergy Clin Immunol 116: 893-9; Rispens, Derksen et al. PLoS One 8: e55566; Guhsl, Hofstetter et al. (2015) Allergy 70: 59-66]. The data suggests that these and likely other interfering agents from the serum bind and saturate the allergen-coated immunoassay surface and although are not detected, suppress the binding and detection of the target sIgE. This binding capacity problem of multiplex assays is exacerbated especially in allergy testing since the standard practice is to use whole food extracts as the antigen on the assay surface (since not all allergenic proteins have been identified). Since whole food extracts can contain hundreds to thousands of proteins, many of which are irrelevant (not allergens), the amount of actual available allergen and hence the surface binding capacity for actual sIgE is further reduced. The ImmunoCAP® assay avoids such problems by using an ultra-high capacity cellulose fiber immunoassay surface, which is not readily saturated with interfering agents like the Luminex® microspheres are. However, the ImmunoCAP® approach is not amenable to miniaturization and multiplexing.

Furthermore, the aforementioned mode of matrix interference (competition from non-IgE immunoglobulins), and other non-specific modes of the matrix effect (see FIG. 1.1-1.4B for example possibilities), vary by patient (i.e. are not a constant). This is shown by the lack of linear correlation with the ImmunoCAP® assay when PC-PURE is not used for AllerBead, in contrast to the excellent linear correlation when PC-PURE is used (see regression plots in FIG. 7 for example; Pearson correlation with ImmunoCAP® averages 0.90 for AllerBead with PC-PURE versus 0.61 without; see also FIG. 6b for Pearson values per each food).

Finally, Table 1 summarizes additional key figures of merit determined for AllerBead with PC-PURE, relative to the gold-standard ImmunoCAP®. Of note, AllerBead (with PC-PURE) could detect ImmunoCAP®-positives as low as 0.10 to 0.26 KIU_{A}/L depending on which food. Sensitivity of AllerBead for all foods was 100% to detect ImmunoCAP®-positives in the range of the maximum measurable by ImmunoCAP® (100 kIU_{A}/L) down to the cutoffs for 95% positive predictive value (PPV) for determining clinical allergy [Sampson and Ho (1997) J Allergy Clin Immunol 100: 444-51; Sampson (2001) J Allergy Clin Immunol 107: 891-6; Perry, Matsui et al. (2004) J Allergy Clin Immunol 114: 144-9], in cases where these cutoffs were available (see Table 1 for further details including the cutoffs, which ranged from 2 kIU_{A}/L to 30 kIU_{A}/L depending on which food). Finally, AllerBead specificity was >94% for all foods.

### Example 5

### Concentrating the Analyte for Improved Diagnostic Sensitivity

In Example 4, patient total IgE was purified using PC-Antibodies but not concentrated (100 μL input serum volume and 100 μL photo-release volume). However, an important advantage of the PC-PURE method is the ability to also concentrate the IgE (or other analyte) before the multiplex immunoassay (or other detection/measurement/quantification method), by photo-releasing in a smaller volume than the input serum sample. Importantly, the PC-PURE method allows the analyte to be concentrated without concentrating the non-target matrix constituents, and hence the interference which arises from them. This is in contrast to non-specific concentrating methods such as ultra-filtration using molecular weight cutoff membranes. To demonstrate the concentrating abilities, PC-PURE and the AllerBead assay were performed as in Example 3 with the following exceptions (see Example 1 for Materials); 46 ImmunoCAP®-annotated food allergy samples were used. To concentrate 5× by volume, the input sample volume used was 500 μL and photo-release volume 100 μL. For comparison to the case where no concentration of the sIgE occurs, identical AllerBead measurements were performed on the same samples where the input volume was 100 μL and photo-release volume remained the same. Scoring cutoffs for determining assay sensitivity were used as described in Example 4.

### Results

In AllerBead, the most important end-point of concentrating the IgE is detection of low-end sIgE positive samples (low-end sensitivity is important as a negative predictor of clinical allergy [Sampson and Ho (1997) J Allergy Clin Immunol 100: 444-51; Sampson (2001) J Allergy Clin Immunol 107: 891-6; Perry, Matsui et al. (2004) J Allergy Clin Immunol 114: 144-9]). FIG. 8 shows sensitivity (percent of ImmunoCAP®-positive patients detected) in the low-end of the ImmunoCAP® scale (defined as between 0.35 kIU_{A}/L and 5 kIU_{A}/L). By concentrating, low-end sensitivity of AllerBead was improved for all foods except milk. Most notably, sensitivity improved 3-fold for peanut, and 2-fold each for egg white and cod (overall, this can be attributed to increased signal-to-noise, which in the entire data set improved on average 2 to 4-fold by concentrating, depending on which food; signal-to-noise was calculated as detailed earlier in the description of FIG. 6A-C). The remaining missed detections of sIgE-positives by AllerBead in comparison to ImmunoCAP® are believed in large part to be related to the use of different allergen extract source material between the two assays and the possible lack of or under-representation of certain allergen proteins in the AllerBead assay. However, it should be noted that blood based sIgE testing is notorious for false positives (relative to clinical allergy) [Sampson and Ho (1997) J Allergy Clin Immunol 100: 444-51; Sampson (2001) J Allergy Clin Immunol 107: 891-6; Perry, Matsui et al. (2004) J Allergy Clin Immunol 114: 144-9; Altmann (2016) Allergo J Int 25: 98-105] (and hence never used alone as a diagnostic), so it is conceivable that the PC-PURE process employed in AllerBead is providing greater specificity and less false-positive detection in the low-end.

### Example 6

### High Capacity NeutrAvidin-Coated Nitrocellulose and PVDF Porous Membrane Microtiter Plates for use in PC-PURE: Binding Capacity Comparison to Commercial Streptavidin Plates

### Materials

NeutrAvidin protein, Biotin-Phycoerythrin (Biotin-PE) and 96-well Streptavidin Coated High Binding Capacity solid polystyrene microtiter plates (hereafter referred to Thermo Streptavidin Plates) were purchased from Thermo Scientific (Waltham, Mass.). 96-well MultiScreenHTS HA Filter Plates, 0.45 μm pore size (nitrocellulose [cellulose nitrate] and cellulose acetate mixed cellulose ester membrane-bottom plates; hereafter referred to as simply “nitrocellulose plates”); and 96-well MultiScreen-IP Filter Plates, 0.45 μm pore size (PVDF/Immobilon-P membrane-bottom plates; hereafter referred to as simply “PVDF plates”) were from EMD Millipore (Billerica, Mass.). See Example 1 for Materials not listed here.

### Preparation of NeutrAvidin-Coated Nitrocellulose and PVDF Plates

Note that nitrocellulose and PVDF plates were microtiter (96-well) filter plates containing a supported porous membrane as the well bottom (and no nitrocellulose or PVDP on the well side-walls). However, these filter plates do not leak without an applied vacuum and were used at all steps in the same manner as standard solid-bottom microtiter plates (i.e. removal of fluids by inversion, aspiration or pipetting), without any filtration through the membrane (vacuum-driven or otherwise).

In the case of PVDF plates, the membrane was first pre-hydrated by adding 100 μL/well of 70% ethanol for 1 minute with shaking. The solution was removed and plates washed 3× 1 min each with shaking in 200 μL/well of purified water. The water was discarded from the wells and the next steps immediately followed.

For both PVDF plates (the pre-hydrated plates were not allowed to dry) and nitrocellulose plates (dry), 50 μL/well of freshly prepared Working NeutrAvidin Solution (1 mg/mL NeutrAvidin in 0.1 M MES, pH 4.7, 0.9% NaCl [154 mM]) was added. Note that as negative controls, wells lacking a NeutrAvidin coaling were also prepared, in which case the NeutrAvidin protein in the Working NeutrAvidin Solution was omitted and replaced with an equivalent amount of BSA protein. Plates were placed on a rotary platform shaker, medium speed, for overnight at +4° C. to allow NeutrAvidin (or BSA) coating by passive adsorption. The solutions were then discarded from the plates which were then washed/blocked at 4× 200 μL/well with 1% BSA (w/v) in TBS for 15 min each, with shaking. The final wash/block solution was discarded and the next steps immediately followed.

### Biotin-Phycoerythrin (Biolin-PE) Binding Assay on NeutrAvidin-Coated Nitrocellulose and PVDF Plates: Comparison to Commercial High Capacity Streptavidin Plates

Biotin-Phycoerythrin (Biotin-PE) was serially diluted to various concentrations in 5% BSA (w/v) in TBS-T. All steps in the dilution series were done in 5% BSA (w/v) in TBS-T. 150 μL/well of each Biotin-PE solution (or blank solution lacking Biotin-PE) was added to the plates. Note that the NeutrAvidin-coated nitrocellulose and PVDF membrane plates were compared to commercial High Capacity Streptavidin Plates from Thermo Scientific (see Materials; hereafter referred to as Thermo Streptavidin plates). The Thermo Streptavidin plates are solid polystyrene plates coated with streptavidin using a proprietary process according to the manufacturer to provide higher binding capacity than other commercially available plates. Biotin-PE binding was allowed to occur in all plate types for 1 hr with mixing on a rotary platform shaker. After capturing the Biotin-PE on the plates, the “Depleted” solutions were removed from the wells and saved. 100 μL/well of each solution was read in a black, solid polystyrene, 96-well microtiter plate using a GloMax® Multimode Microplate Reader (Promega) in fluorescence mode with the proper filter set. In addition to the “Depleted” solutions, the “Input” solutions which never contacted the NeutrAvidin-coated nitrocellulose or PVDF plates or the Thermo Streptavidin plates were also read in the same manner.

### Results

A Biotin-PE standard curve was used to convert the fluorescence values to μg of Biotin-PE. Total binding was calculated as the input minus the Biotin-PE remaining unbound in the corresponding Depicted solution. Specific binding was calculated by correcting for non-specific binding, by subtracting out the binding occurring on the negative control wells which lacked a NeutrAvidin coating (specific binding could not be calculated with Thermo Streptavidin plates since wells produced in the same manner but lacking the streptavidin coating were not available). Results in FIG. 9a show that the high-end binding capacity of the NeutrAvidin-coated nitrocellulose plates was at least 3 to 4-fold better than the commercial Thermo Streptavidin plates. For example, at the highest Biotin-PE input, 65 μg/well, total binding was 21 μg/well and specific binding was 17 μg/well for the NeutrAvidin-coated nitrocellulose. In comparison, total binding on the Thermo Streptavidin plates was 6 μg/well (while specific binding could not be calculated for the Thermo Streptavidin plates, it would only be equal or lower than the total binding).

In a second set of experiments, NeutrAvidin-coated nitrocellulose plates were compared to NeutrAvidin-coated PVDF plates as shown in FIG. 9b. First, the high-end binding capacity of the NeutrAvidin-coated nitrocellulose plates was highly consistent with the prior set of experiments (in FIG. 9a). For example, at the highest Biotin-PE input, 65 μg/well, total binding was 28 μg/well (vs. 27 in prior set of experiments) and specific binding was 16 μg/well (vs. 17 in prior set of experiments). Binding capacity of the NeutrAvidin-coated PVDF plates was comparable to the nitrocellulose plates. For example, at the highest Biotin-PE input, 65 μg/well, total binding was 30 μg/well and specific binding was 13 μg/well.

It should be noted that with both the nitrocellulose and PVDF plates, not surprisingly, non-specific binding was significant at the highest Biotin-PE input (65 μg/well) but subsided at the lower inputs (i.e. total binding and specific binding were similar; specific binding could not be calculated with the Thermo Streptavidin plates as noted earlier).

Finally, in a third set of experiments, Biotin-PE binding time on NeutrAvidin-coated nitrocellulose plates and the commercial Thermo Streptavidin plates was tested. While prior experiments used 1 hr binding time, this set of experiments compared 1 hr and overnight binding times (roughly 18 hours). The maximum specific Biotin-PE binding on the NeutrAvidin-coated nitrocellulose plates at the highest input of 79 μg/well was increased 3-fold to approximately 60 μg/well with overnight binding (versus 20 μg/well at 1 hr). Conversely, the Thermo Streptavidin plates were not improved at all by overnight binding, with the maximum binding remaining far below that of the NeutrAvidin-coated nitrocellulose plates.

### Example 7

### High Capacity Porous Membrane Microtiter Plates for PC-PURE: Application to Multiplex Blood-Based Allergy Assays (AllerBead Assay)

### PC-Antibody Coating of NeutrAvidin-Coated PVDF and Nitrocellulose Plates

See Examples 1 and 6 for Materials, Nitrocellulose and PVDF membrane-bottom plates coated with NeutrAvidin were prepared as in Example 6. Subsequent coating of these plates, and also the commercial Thermo Streptavidin plates (see Example 6), with the PC-Antibody immediately followed: As noted in Example 6, the nitrocellulose and PVDF porous membrane based plates, although they are filter plates, were used at all steps in the same manner as standard solid-bottom microtiter plates (i.e. removal of fluids by inversion, aspiration or pipetting), without any filtration through the membrane (vacuum-driven or otherwise). The Thermo Streptavidin plates are not filter plates (solid polystyrene), and thus were also used in this manner. The PC-Biotin conjugated anti-IgE photocleavable antibody (PC-Antibody) was prepared as in Example 1. 50 μL/well of Working PC-Antibody Solution (0.25 μg/μL PC-Antibody in 0.1% BSA [w/v], TBS) was added to the plates for 1 hr with shaking. Plates were then washed 4× 200 μL/well for 5 min each with 0.1% BSA (w/v) in TBS followed by rinsing 4× 200 μL briefly in purified water. The water was discarded from the plates and the plates then dried overnight in a chemical fume hood, with the plate uncovered and the blower of the hood on (the drying is necessary for better storing and shipping of the plates in a commercial setting). The resultant plates are hereafter referred to as Nitrocellulose PC-Plates, PVDF PC-Plates, or Thermo PC-Plates, corresponding to the nitrocellulose membrane, PVDF membrane and the Thermo polystyrene based plates, respectively. Plates were used after drying or stored sealed in zip-top plastic bags with desiccant pouches, at +4° C. and protected from light.

### PC-PURE of IgE and Multiplex Immunoassays of Allergen-Specific IgE (AllerBead Assay)

PC-PURE of IgE from samples using agarose beads coated with an anti-IgE PC-Antibody (collectively referred to as PC-Beads) followed by multiplex microsphere-based immunoassay of allergen-specific IgE (sIgE), referred to as the AllerBead assay, were performed as in Example 4. This same process was also done using the aforementioned Nitrocellulose PC-Plates, PVDF PC-Plates, and Thermo PC-Plates in the same manner as with the PC-Beads with the following exceptions: The aforementioned PC-Plates were used for IgE purification by PC-PURE instead of PC-Beads. As noted above and in Example 6, the nitrocellulose and PVDF porous membrane based plates, although they are filter plates, were used at all steps in the same manner as standard solid-bottom microtiter plates (i.e. removal of fluids by inversion, aspiration or pipetting), without any filtration through the membrane (vacuum-driven or otherwise). Before use, the dry PC-Plates were re-hydrated by washing 4× 200 μL/well briefly with TBS-T. The input sample volume (per well) was 100 μL, as with the PC-Beads, but IgE capturing in the PC-Plates was done at 37° C. Washing after IgE capturing was with TBS-T, 4× 200 μL/well briefly and then 3× 200 μL/well for 10 min each with shaking. The photo-release volume was 25 μL (and as with the PC-Beads, the “Combined Method” of photo-release was used, in this case, by placing the allergen-coated Luminex® immunoassay microspheres into the wells of the PC-Plates during the photo-release). Following photo-release, the incubation was for 1 hour with shaking in order to allow the photo-released complexes to bind to the allergen-coated Luminex® immunoassay microspheres (and this incubation occurred still within the photocleaved PC-Plates). Microspheres were then recovered from the photocleaved PC-Plates and processed on the MagMAX™ robot as done for the experiments in Examples 1 and 4, to complete the remainder of the AllerBead assay.

### Results

24 serum samples were analyzed using the PC-Beads, Nitrocellulose PC-Plates or Thermo PC-Plates for PC-PURE of IgE followed by the multiplex microsphere-based AllerBead immunoassay of allergen-specific IgE (sIgE). The multiplex AllerBead assay yielded 168 data points (24 samples each tested against 7 food allergens in the multiplex assay [food extracts only]—peanut, shrimp, cashew, egg white, cod, wheat and soy). In addition, each sample was analyzed for sIgE positivity or negativity for all food allergens under study using the gold-standard, FDA-cleared, non-multiplex ImmunoCAP® test. To calculate the AllerBead signal-to-noise, the AllerBead result for each ImmunoCAP®-positive data point (i.e. each ImmunoCAP®-positive sample-allergen pair) was divided by the average background, defined as the average AllerBead result for all ImmunoCAP®-negatiye data points for the corresponding food allergen. AllerBead signal-to-noise ratios were then averaged for each food allergen and the data graphed in FIG. 10a (Table 2 lists the number of ImmunoCAP®-positive and negative data points for each food allergen). Results show that the Nitrocellulose PC-Plates performed comparable or better than the PC-Beads for every food allergen except for peanut, where the PC-Beads yielded an average signal-to-noise ratio of approximately 2-fold better. Conversely, the Thermo PC-Plates yielded average signal-to-noise ratios that were 3 to 30-fold worse than the Nitrocellulose PC-Plates depending on which food allergen. Table 2 shows the Pearson con-elation (r value) between the PC-Bead, Nitrocellulose PC-Plate or Thermo PC-Plate based multiplex AllerBead method and the gold-standard, FDA-cleared, non-multiplex ImmunoCAP® method, for this 24 sample cohort. Notably, both the PC-Bead and Nitrocellulose PC-Plate based AllerBead methods in general correlated strongly with ImmunoCAP® (average Pearson's r for all food allergens of 0.85 and 0.94, respectively), whereas the Thermo PC-Plate based AllerBead method correlated poorly with ImmunoCAP® (average Pearson's r of 0.53).

Critically, while the superior high-end binding capacity of the nitrocellulose plates (see Example 6) contributes to their improved performance (versus the Thermo Scientific plates) in PC-PURE followed by the AllerBead assay, other factors are also involved. First, entrapment of the captured analyte (IgE) within the 3-dimensional pores of the nitrocellulose membrane (Nitrocellulose PC-Plates), among a high density of immobilized binding agent (PC-Antibody), may reduce the effective “off-rate” of the captured analyte (e.g. during subsequent washing steps) compared to the more 2-dimensional surface of the solid polystyrene Thermo Scientific plates (Thermo PC-Plates). Furthermore, because on the Nitrocellulose PC-Plates the binding agent (PC-Antibody) is focused at high density only on the well bottoms, the analyte is more effectively concentrated by photo-releasing in a smaller volume compared to the input sample volume (in this Example, 100 μL input sample and 25 μL photo-release volume for all plate types). This contrasts with the Thermo Scientific plates (Thermo PC-Plates), which not only have lower binding capacity (see Example 6), but this binding capacity is distributed over the entire surface of the well (bottoms and side-walls, to the 100 μL level according to the manufacturer). Therefore, photo-releasing in a smaller volume (e.g. 25 μL) and recovering ail the photo-released material is much less efficient in the Thermo PC-Plates.

In another experiment, PC-PURE followed by AllerBead was compared using the anti-IgE photocleavable antibody (PC-Antibody) on NeutrAvidin-coated nitrocellulose membrane-bottom microtiter plates (Nitrocellulose PC-Plates) and on NeutrAvidin-coated PVDF membrane-bottom microtiter plates (PVDF PC-Plates). An analysis of 16 serum samples and 8 food allergens (peanut, milk, shrimp, cashew, egg white, cod, wheat and soy) was performed in this case. A regression plot between the two plate types of the MFI (Median Fluorescence Intensity), the raw output of the AllerBead assay, is shown in FIG. 10b for all data points (all samples and all food allergens [food extracts only]; 128 data points total). The results from the PVDF and Nitrocellulose PC-Plates were highly comparable with the exception of 2 outlier data points. Overall, the slope of the linear regression line was 1.3 and Pearson's r correlation 0.95, showing excellent agreement. This is explained by the common desirable features of both the Nitrocellulose and PVDF PC-Plates: Both have a similarly high binding capacity, as in Example 6, and both have a high capacity porous membrane containing a high density of binding agent only on the well bottoms (allowing for efficient concentration of the analyte). Compatibility of the membranes with photo-release is also an important trait, such as translucency, at least when welted, and a thin enough membrane, 150 μm in the case of the Nitrocellulose PC-Plates, such that sufficient light is delivered. Finally, it should be noted that in comparison to porous gels, porous membranes have the advantage that they are easier to store and handle. For example, unlike gels, membranes will no shrink, crack or become brittle when dried (such as for storage purposes) and are more structurally rigid and less fragile than gels (less likely to become damaged or break apart during processing and manipulation).

### Example 8

### Nitrocellulose Membrane Coating of Solid Microliter Plates; Application to PC-PURE and Microsphere-Based Immunoassays of Allergen-Specific IgE (AllerBead Assay)

### Materials

The recombinant Der p 2 (rDer p 2.0101) dust mite allergen protein and the monoclonal humanized chimeric IgE anti-Der p 2 antibody (sub-standardized to WHO IgE 75/502) were from Indoor Biotechnologies (Charlottesville, Va.), WebSeal Plate+ 96-Well Glass-Coated Solid Polypropylene Microplates (microtiter plates) and Nitrocellulose Membrane Sheets (0.45 μm pore size) were from Thermo Scientific (Waltham, Mass.). See Examples 1 and 6 for other Materials not listed here.

### Preparing Custom Cast Nitrocellulose Membranes in Solid Microtiter Plates

The nitrocellulose solution itself was prepared similar to published reports [Flynn, Arndt et al. (2013) Advances in Chemical Science 2: 9-18]: Commercially available nitrocellulose membrane sheets (see Materials) were cut into small pieces and dissolved into a solution comprised of 5.771 mL of 100% acetone, 4.206 mL of 100% ethanol, and 415 μL purified water. Only after the nitrocellulose membrane was fully dissolved, 594 μL more of purified water was added. The final concentration of nitrocellulose was 34 mg/mL and 85 mg/mL.

Into each well of a glass-coated solid polypropylene microtiter plate (see Materials), 25 μL of the prepared nitrocellulose solutions was added and the plates dried for 1 hour under vacuum. Note that the glass-coated polypropylene plates were found to have more desirable properties compared to uncoated polypropylene with respect to better spreading of the added nitrocellulose solutions and better adhesion of the formed nitrocellulose membrane, likely due to the more hydrophilic properties of the glass. Polypropylene or glass-coated polypropylene plates were chosen due to better solvent resistance compared to polystyrene.

### PC-PURE of IgE and Microsphere-Based Immunoassays of Allergen-Specific IgE (AllerBead Assay)

Performed as with the Nitrocellulose PC-Plates in Example 7, with the following exceptions: Instead of serum, the sample was comprised of monoclonal humanized chimeric IgE anti-Der p 2 antibody (the allergen-specific IgE [sIgE]) in BSA Block buffer and only one species of allergen-coated Luminex® microspheres was used for AllerBead, containing the recombinant Der p 2 (rDer p 2.0101) dust mite allergen protein (prepared to 1.25 mg/mL in PBS with 5 mM EDTA for coupling to the microspheres).

### Results

For the custom cast nitrocellulose membrane coated plates, nitrocellulose solutions were added to glass-coated solid polypropylene microtiter plates and dried to form the porous membrane. Small volumes of nitrocellulose solutions (25 μL in this Example) were used to ensure the membrane formed on or near the well bottoms such that concentrating the analyte upon photo-release (during PC-PURE) was more efficient (such as by inputting larger sample volume compared to the photo-release volume, 100 μL and 25 μL in this Example, respectively). The image in FIG. 11a shows that the nitrocellulose membrane forms a ring morphology around the well bottoms at both nitrocellulose concentrations tested (some also on the well side walls, but not higher than the 25 μL level), with the 34 mg/mL nitrocellulose solution forming a thinner and somewhat less uniform membrane.

Next, monoclonal humanized chimeric IgE anti-Der p 2 antibody was spiked into solutions and then PC-PURE purified, using both the custom cast nitrocellulose-coated solid microtiter plates (prepared in this Example) and commercially available nitrocellulose-bottom filter plates (see Example 7). Both plate types were coated with NeutrAvidin and then the anti-IgE PC-Antibody for this purpose (as done in Example 7), and hereafter referred to in this Example as “Custom Nitrocellulose PC-Plates” and “Commercial Nitrocellulose PC-Plates”, respectively. The AllerBead assay followed PC-PURE. FIG. 11b shows a plot of the Median Fluorescence Intensity (MFI), the raw output of the AllerBead assay, versus the various concentrations of input monoclonal humanized chimeric IgE anti-Der p 2 antibody. For the Custom Nitrocellulose PC-Plates, data are shown for the plates prepared using the 34 mg/mL nitrocellulose solution, since in the 85 mg/mL condition, sporadic results were believed to be the result of part or all of the membrane in some wells becoming detached during processing of the assay. However, it should be noted that different plate surface chemistries will be employed to ensure a stronger and more stable attachment of the nitrocellulose membrane to the plate (e.g. epoxy silane treatment of the glass-coated plates prior to forming the nitrocellulose membrane). Nonetheless, the results in FIG. 11b show that the 34 mg/mL Custom Nitrocellulose PC-Plates perform comparably to the Commercial Nitrocellulose PC-Plates, albeit with a slightly lower signal (Custom Nitrocellulose PC-Plates yield signals that are 30% lower on average).

### Example 9

### Comparison of Finger-Stick Capillary Serum to Venous Draw, and Room Temperature Serum Storage to Storage Frozen; PC-PURE IgE Purification Followed by Multiplex Blood-Based Allergy Assays (AllerBead Assay)

### Results

PC-PURE IgE purification followed by the multiplex AllerBead assay for quantification of allergen-specific IgE (sIgE) to food allergens was performed as in Example 4 with the following exceptions: Blood collected by venipuncture into clot-activating BD (Becton Dickinson) Vacutainers® (Becton Dickinson, Franklin Lakes, N.J.) and converted to serum using conventional methods was compared to matching capillary blood from the same patients collected by finger-stick using BD Contact-Activated Lancets (High Flow; Blue) into clot-activating BD Microtainers® (for serum conversion according to manufacturer's instructions). In this case, 50 μL of serum was input into the PC-Beads for PC-PURE of IgE and the photo-release volume was also 50 μL for the subsequent AllerBead assay. Furthermore, in separate experiments, room temperature storage (10 days) of venous derived serum was compared to normal storage conditions (frozen) of venous derived serum (aliquots of same samples) prior to PC-PURE and the AllerBead assay. In this case, 100 μL of serum was input, into the PC-Beads for PC-PURE of IgE and the photo-release volume was also 100 μL for the subsequent AllerBead assay. 8 and 14 food allergic patients spanning a range of sIgE positivity and negativity (as determined by analysis using the gold-standard, FDA-cleared, non-multiplex ImmunoCAP® test) to the food allergens under study (see Example 4) were used for the venous draw versus finger-stick study and the room temperature serum storage versus frozen storage study, respectively.

Results in FIG. 12a show a regression plot of the Median Fluorescence Intensity (MFI), the raw output of the AllerBead assay, for all data points (all food allergens [8 food extracts only] and all 8 samples), comparing finger-stick derived serum to venous derived serum. A high correlation was observed between the two blood collection methods, with a Pearson's r correlation of 0.99 and slope of the linear regression line of 0.88 (finger-stick on Y-Axis). Note that validation of PC-PURE and AllerBead using the conventional venous derived serum method, in reference to the gold-standard, FDA-cleared, non-multiplex ImmunoCAP® test, was achieved in Example 4.

Likewise, results in FIG. 12b show a regression plot of the Median Fluorescence Intensity (MFI), the raw output of the AllerBead assay, for all data points (ail food allergens [8 food extracts only] and all 14 samples), comparing room temperature stored venous derived serum (10 days) to the conventional method of serum stored frozen (aliquots of the same samples). A high correlation was observed between the two serum storage method methods, with a Pearson's r correlation of 0.98 and slope of the linear regression line of 1.26 (room temperature storage on Y-Axis). Note that validation of PC-PURE and AllerBead using the conventional method of serum stored frozen, in reference to the gold-standard, PDA-cleared, non-multiplex ImmunoCAP® test, was achieved in Example 4.

### Example 10

PC-PURE of Analytes (Target Proteins/Biomarkers) using Photocleavable Aptamers (PC-Aptamers) Followed by Downstream Immunoassay.

### Materials

Aptamers were obtained from Aptamer Sciences (South Korea) and Base Pair Biotechnologies (Pearland, Tex.) and were synthesized with a 5′ photocleavable biotin (PCB) using AmberGen's (Watertown, Mass.) PC-Biotin Phosphoramidite reagent (distributed by Glen Research, Sterling, Va.). See Examples 1 and 6 for Materials not listed here. Recombinant “Target Proteins” (Analytes/Biomarkers) were from R&D systems (Minneapolis, Minn.) and Abcam (Cambridge, Mass.); these proteins were EGFR, HGFR/C-Met, VEGFR and AKT2. Microsphere-based multiplex-compatible Luminex® sandwich immunoassay kits for measuring the Target Proteins were from R&D Systems (Minneapolis, Minn.) and EMD-Millipore (Billerica, Mass.).

### Photocleavable Aptamer (PC-Aptamer) Preparation

5′ PC-Biotin labeled aptamers (PC-Aptamers) were re-folded fresh the day of use by first preparing the PC-Aptamer to a concentration of 17 μM in PBS which was supplemented with 1 mM (final) MgCl₂, then by heating to 95° C. for 5 minutes and then allowing it to cool to room temperature for 15 min. After the PC-Aptamer had cooled, it was supplemented with 0.05% (v/v) final Tween-20 concentration. It was then further diluted to 0.4 μM PC-Aptamer using PBS-MT (PBS with 1 mM MgCl₂ and 0.05% Tween-20 [v/v]) to create the Working PC-Aptamer Solution.

### Attaching PC-Aptamer to Streptavidin Plates

To load the PC-Aptamer onto Thermo Streptavidin Plates (see Example 6 for plates), plates were first washed 4× 200 μL/well briefly, using PBS-MT. Plates were then coated with 50 μL of 0.4 μM Working PC-Aptamer Solution, for a ratio of 20 pmoles of PC-Aptamer/well, and incubated for 30 min with medium shaking. Plates (hereafter referred to as PC-Aptamer Plates) were then washed 5× 200 μL/well in PBS-MT.

### Target Protein (Analyte/Biomarker) Capture, Photo-Release and Multiplex Microsphere-Based Sandwich Immunoassay

“Plus Target Protein” solutions were prepared by diluting the recombinant Target Protein (EGFR, HGFR/C-Met, VEGFR, AKT2) to the appropriate working concentration using Aptamer Block Buffer (PBS-MT supplemented with BSA to 1% [w/v]final). “Minus Target Protein” solution was simply Aptamer Block Buffer alone. In some cases, serum samples with and without the Target Protein spiked in were used instead. To perform Target Protein capture on PC-Aptamer Plates, plates were pre-washed 4× 200 μL/well in Aptamer Block Buffer. After the last wash was discarded, 50 μL/well of Plus Target Protein or Minus Target Protein solution was added to the PC-Aptamer Plates, and also to Negative Control wells (wells not coated with PC-Aptamer). Plates were shaken for 1 hour to allow capture of the Target Protein. PC-Aptamer wells were next washed in BSA Block (1% BSA [w/v] in TBS-T) for 3× 10 min with shaking, using 200 μL/well each wash. Wells were next filled with 50 μL of BSA Block to maintain the volume integrity. Luminex® microspheres (from Luminex® kits; see Materials), which contained capture antibodies directed against the Target Proteins for multiplex-compatible sandwich immunoassay, were diluted 1/20 using BSA Block before adding 50 μL to each well. This resulted in 100 μL for each well (overall ½× dilution of the samples destined for photo-release; same dilution as the other samples not in the plates such as detailed later). Photo-release was performed as previously described in Example 1 (“Combined Method”). Following photo-release, 50 μL from each well (including suspended microspheres) was transferred to a plain 96-well microtiter plate for the Luminex® microsphere-based sandwich immunoassay. In separate empty wells of the microtiter plate, 25 μL of the Luminex® microspheres (diluted from 1/20 as detailed earlier) were combined with 25 μL of Calibration Standards (from Luminex® kits; see Materials) and Input Samples (Plus and Minus Target Protein solutions which never contacted the PC-Aptamer plates). Luminex® microspheres were incubated for 2 hours with shaking to allow the Target Protein to be captured onto the Luminex® microspheres via the attached capture antibody. Manufacturer instructions for the Luminex® immunoassay kits were followed to complete the procedure, which included addition of a detection antibody to complete the sandwich immunoassay. A magnetic separator (a magnetic slab which attaches underneath the microtiter plate) was used to immobilize the magnetic microspheres on the plate bottom during fluid removal from the wells for these steps. Assay readout was performed in a Luminex® MagPix® instrument.

In some cases the PC-Aptamer was loaded onto streptavidin agarose heads instead of the Thermo Streptavidin Plates and used for the PC-PURE steps. In this case, other than this change, the procedure followed similarly except that the agarose beads were processed in micro-centrifuge filter units or microtiter filter plates (see Example 1 Materials) to execute PC-PURE. Note that during the subsequent Luminex® microsphere-based immunoassay steps, the processing of the Luminex® microspheres with the magnetic separator as described earlier allows the non-magnetic agarose beads (photocleaved PC-Beads) to be washed away (which are no longer needed after the photo-release step).

### Results

The ability of the PC-Aptamers to capture and photo-release the Target Proteins was first validated. This was accomplished by using a model system comprised of recombinant Target Proteins spiked into a buffer solution. Four cancer biomarkers (VEGFR, HGFR, EGFR and AKT2) each in plain buffer (16 ng/mL, 10 ng/mL, 20 ng/mL and 300 ng/mL, respectively) were subjected to PC-PURE using the PC-Aptamer Plates. The “Input” sample is the solution prior to isolation on the PC-Aptamer Plates. The “Photo-Release” fraction is the solution after elution from the PC-Aptamer plates using UV light treatment. The “Input” samples as well as the “Photo-Release” sample fractions were measured by a sandwich immunoassay on the multiplex Luminex® platform (the PC-Aptamer is used only for Target Protein purification, and although present, does not participate in the Luminex® immunoassay that follows), “Blank” is synonymous with “Minus Target Protein” and indicates where the initial Input lacked the Target Protein and in all other cases the initial Input contained the Target Protein. FIG. 13a summarizes the results of this PC-Aptamer validation showing that all 4 PC-Aptamers could specifically capture and photo-release the Target Proteins which could subsequently be measured on the multiplex Luminex® immunoassay platform. Overall recovery of the Target Proteins (with purifying but not concentrating by PC-PURE) ranged from 20-78%.

PC-PURE with PC-Aptamers was also evaluated on serum. The entire process of PC-PURE (using PC-Aptamers on agarose beads in this case) coupled with subsequent Luminex® immunoassay was performed on the VEGFR biomarker spiked into serum (at various concentrations) and compared to “Standard Luminex®” assays (i.e. direct immunoassay without PC-PURE—no purifying or concentrating). In the case of PC-PURE, the VEGFR Target Protein was concentrated 8-fold by volume (400 μL input sample and 50 μL photo-release volume). As shown in FIG. 13b, comparing Standard Luminex® assays of VEGFR in crude serum versus in plain buffer (BSA Block), showed a substantial matrix effect in the form of significantly decreased sensitivity (up to 10-fold). Conversely, the use of PC-PURE to both purify and concentrate the VEGFR produces a marked improvement in sensitivity (up to 11-fold).

### Example 11

Dual-Labeled Photocleavable & Fluorescent Binding Agents: Integrating PC-PURE with Downstream Detection

### Materials

The anti-human TIMP-1 antibody, recombinant human TIMP-1, the human TIMP-1 ELISA and the human TIMP-1 Luminex® microsphere-based sandwich immunoassay kit were from R&D systems (Minneapolis, Minn.). The Lightning Link® R-Phycoerythrin Conjugation Kit was from Innova Biosciences (Cambridge, UK). See Examples 1, 6 and 10 for any Materials not listed here.

### Dual Photocleavable-Biotin (PC-Biotin) and Phycoerythrin (PE) Labeling of an Antibody: Dual-Labeled PC-Antibody

The anti-TIMP antibody (40 μg in 100 μL of PBS) was supplemented to 100 mM sodium bicarbonate from a 1M stock. 15 molar equivalents of the PC-Biotin-NHS labeling reagent were immediately added (from a 50 mM stock in anhydrous DMP) to the antibody. The reaction was carried out for 30 min with gentle mixing. To remove unreacted PC-Biotin-NHS reagent, the reaction mix was desalted on a PD SpinTrap G-25 spin column, performed according to the manufacturer's instructions (equilibration and elution in PBS). Following desalting, the final product corresponding to the PC-Biotin labeled anti-TIMP antibody (PC-Antibody) was supplemented with ¼ volume of5× concentrated PBS to ensure adequate buffering capacity.

Next, for Phycoerythrin (PE) Labeling, the Lightning Link® R-Phycoerythrin Conjugation Kit was used according to the manufacturer's instructions. Specifically, 1 μL of LL-modifier for each 10 μL of PC-Antibody volume was added and mixed well. This solution was then added to one vial of LL-mix, directly onto the lyophilized pink material and resuspended gently by pipetting. The vial was then protected from light by covering with foil and incubated for 3 hours at room temperature, or overnight at 4° C. Following incubation, LL-Quencher reagent was added to the PC-Antibody solution (at a ratio of 1 μL to 10 μL of PC-Antibody solution). This mixture was then incubated at room temperature for 30 minutes. The final product corresponding to the dual-labeled PC-Biotin R-Phycoerythrin labeled Anti-TIMP antibody (hereafter referred to as PCB-PE-Anti-TIMP Antibody) was aliquoted and stored at −70° C.

Loading the PCB-PE-Anti-TIMP Antibody onto streptavidin beads (to create the PC-Beads) was performed as previously described for the PC-Antibody in Example 1. PC-PURE of TIMP using PC-Beads followed by TIMP measurement on a multiplex Luminex® microsphere-based immunoassay was also performed as described in Example 1, with the following exceptions: The analyte was recombinant human TIMP at 0.25 ng/mL in BSA Block. The Luminex® microsphere-based immunoassay used a commercially available kit (see Materials) essentially according to the manufacturer's instructions with the following exceptions: Following the photo-release from the PC-Beads (“Combined Method” as detailed in Example 1) and re-capture of photo-released [PCB-PE-Anti-TIMP Antibody]-[TIMP] complexes onto the anti-TIMP antibody-coated microspheres, the use of the detection reagents prescribed in the commercial Luminex® microsphere-based immunoassay kit was omitted (since the photocieaved PCB-PE-Anti-TIMP Antibody was used for detection). Instead, microspheres were simply washed in TBS-T and re-suspended in 100 μL of TBS-T for readout in a Luminex® MagPix® instrument.

In some cases, PC-PURE was performed using the PCB-PE-Anti-TIMP Antibody on Thermo Streptavidin microtiter plates instead of on PC-Beads (see Examples 6 and 7 for methods; 0.5 μg PCB-PE-Anti-TIMP Antibody per well in this case). In other cases, PC-PURE of TIMP was not performed and the PCB-PE-Anti-TIMP Antibody was simply used for detection in the Luminex® microsphere-based immunoassay kit (again, replacing the kit detection reagents). In other cases, TIMP was instead quantified using a commercial ELISA assay (see Materials).

### Results

This Example describes the development of dual-labeled PC-Antibodies that carry both a photocleavable biotin (PCB) for PC-PURE and a fluorescent label (phycoerythrin [PE] in this Example) for readout in the downstream multiplex immunoassay. Without dual-labeled PC-Antibodies, PC-PURE followed by downstream sandwich immunoassay would require three antibodies bound to each analyte (PC-Antibody for PC-PURE as well as capture and detection antibodies for the sandwich immunoassay). However, this approach has major limitations including: i) the difficulty in finding and validating three immunoassay-quality antibodies against three different epitopes on each analyte. In particulars “steric hindrance” makes it difficult for three large (150 kDa) antibody molecules to simultaneously bind to an analyte as compared to two used in standard sandwich immunoassays; and ii) significant added cost of using three antibodies per analyte for the total assay. These problems have been overcome by developing dual-labeled PC-Antibodies, since the PC-Antibody also serves as the detection antibody in the downstream sandwich immunoassay (reducing the antibody requirement back to two total for the overall process).

The first step in this Example, using TIMP as the model analyte (biomarker), was to prepare an anti-TIMP photocleavable antibody dual-labeled with PC-Biotin and R-Phycoerythrin (PCB-PE-Antibody) which was suitable for PC-PURE of TIMP prior to its input into a solid-phase immunoassay for quantification. First, it was verified that dual-labeled PCB-PE-Antibodies could isolate the analyte for the initial step in PC-PURE. Example results for the TIMP analyte are shown in FIG. 14a. For this, a dual-labeled PCB-PE-Anti-TIMP antibody was used on the Thermo Streptavidin microtiter plates for isolating TIMP protein. The amount of free TIMP was quantified by ELISA in the Input solution (solution prior to isolation) and Depleted fraction (solution after isolation). As shown in FIG. 14a, the PCB-PE-Anti-TIMP antibody depleted (bound) 100% of the detectable TIMP from the input solution, whereas if the PCB-PE-Anti-TIMP antibody was omitted from the microtiter plates, there was no non-specific TIMP depletion.

Next it was verified that the PCB-PE-Anti-TIMP antibody could effectively act as a detection antibody in the downstream Luminex® microsphere-based multiplex sandwich immunoassay. In this Example, a commercial multiplex-compatible Luminex® sandwich immunoassay kit for TIMP was used. The normal detection system in the kit, which uses a biotinylated detection antibody followed by a streptavidin-PE conjugate, was compared to using only the PCB-PE-Anti-TIMP Antibody for detection in the Luminex® assay without streptavidin-PE. As shown in FIG. 14b, the PCB-PE-Anti-TIMP antibody was equally effective in detection as the standard system provided in the commercial kit (to demonstrate detection abilities, the raw Median Fluorescence Intensity [MFI] of the Luminex® assay is shown in FIG. 14b).

Finally, FIG. 14c shows data from the entire process of PC-PURE (isolation of analytes from buffer using a PCB-PE-Anti-TIMP Antibody, on agarose beads in this case [PC-Beads], followed by photo-release) and sandwich immunoassay formatted on the multiplex Luminex® platform. An overall recovery of 41% of the TIMP biomarker was observed (note that PC-PURE was used to purify but not concentrate the analyte in this Example). Minus UV negative controls demonstrate the specificity (light-dependency) of PC-PURE (samples subjected to PC-PURE except UV treatment omitted during photo-release step—showing no detectable TIMP in the subsequent immunoassay). Blank samples lacking TIMP also demonstrate the specificity of the assay.

### Example 12

### High Capacity NeutrAvidin-Coated Nitrocellulose Microtiter Plates for use in PC-PURE: Direct Versus Indirect Coating of the Nitrocellulose

### Materials

Pierce™ Bovine Serum Albumin, Biolinylated (Biotin-BSA) was obtained from Thermo Scientific (Waltham, Mass.). See Examples 1 and 6 for Materials not listed here.

### Preparation of NeutrAvidin-Coated Nitrocellulose

Direct coating of NeutrAvidin onto the nitrocellulose membrane of the microtiter plates (referred to as nitrocellulose plates) was performed as in Example 6. Indirect coating was performed as follows: 50 μL/well of freshly prepared 1 mg/mL Biotin-BSA in MES Buffer was added to the nitrocellulose plates and the plates shaken for overnight at +4° C. to allow coating of the Biotin-BSA (by passive adsorption) onto the nitrocellulose membrane. The Biotin-BSA solution was then removed from the wells and the plates washed/blocked 4× 200 μL/well with 1% BSA (w/v) in TBS for 15 min each wash with shaking. The plates were then coated with 1 mg/mL NeutrAvidin in 1% BSA (w/v) in TBS at 50 μL/well for 1 hr with mixing. Since NeutrAvidin is a tetramer, with 4 biotin-binding sites per molecule, the attachment of NeutrAvidin to the Biotin-BSA coated surface still leaves sites remaining for further biotin binding. The NeutrAvidin solution was then removed from the wells and the plates again washed/blocked 4× 200 μL/well with 1% BSA (w/v) in TBS for 15 min each wash with shaking.

Biotin-Phycoerythrin (Biotin-PE) Binding Assay on Nitrocellulose Plates Directly and Indirectly Coated with NeutrAvidin

Performed as in Example 6.

### Results

Data was analyzed as in Example 6 (input Biotin-PE amount per well plotted versus bound Biotin-PE amount; note that constant volumes of 150 μL were added per well for ail amounts, therefore variable concentrations of Biotin-PE input were used). In this Example, Biotin-PE binding was compared for the nitrocellulose plates, directly and indirectly coated with NeutrAvidin, and for the commercially available Thermo Streptavidin plates (see Example 6 for details on Thermo Streptavidin plates). Note that data shown in this Example for the nitrocellulose plates has been corrected for non-specific Biotin-PE binding (see Example 6 for details).

While Example 6 showed that directly coated NeutrAvidin Nitrocellulose plates have a substantially higher maximum Biotin-PE binding capacity compared to the Thermo Streptavidin plates (confirmed in this Example, see FIG. 15a), the expanded Biotin-PE dilution series used in this Example shows that the binding efficiency of the directly coated NeutrAvidin Nitrocellulose plates is inferior to the Thermo Streptavidin plates at the lower concentrations, starting at 3.75 μg/well of Biotin-PE input (25 μg/mL) and lower (see FIGS. 15a and 15b). This could be explained by partial denaturation of the NeutrAvidin upon direct passive adsorption to the (hydrophobic) nitrocellulose, thereby decreasing its binding affinity for biotin, and/or increased steric hindrance (to ligand binding) when NeutrAvidin is directly adsorbed to the nitrocellulose, which could again affect its binding efficiency for biotin. These effects would likely manifest at lower Biotin-PE concentrations (lower input amounts), when the binding capacity of the plates is not exceeded. Indeed, the indirect coating of the nitrocellulose plates with NeutrAvidin solves this problem, with the low-end binding efficiency (again at 3.75 μg/well and lower) essentially matching that of the Thermo Streptavidin plates (see FIG. 15b which is a line plot of the mid to low-range of Biotin-PE input amounts). It is worth noting that ail plate types show a multi-phasic binding response as a function of the Biotin-PE input (in particular the Thermo Streptavidin plates), indicating it is a complex system with multiple factors at play (FIG. 15b). Nonetheless, a linear range (input versus bound Biotin-PE) can be found for all three plate types, with linear regression R² values >0.99 in all cases (FIG. 15c). The Thermo Streptavidin plates perform well in the low-end, with the linear range extending from 0-2 μg/wel of Biotin-PE input, whereas the directly coated NeutrAvidin nitrocellulose plates perform well in the high-end, with a linear range from 2-15 μg/well of Biotin-PE input. Lastly, the indirectly coated NeutrAvidin Nitrocellulose plates match the Thermo Streptavidin in the low-end, but perform better in the high-end, with a linear range from 0-7.5 μg/well of Biotin-PE input.

### DESCRIPTION OF THE DRAWINGS

FIG. 1.1-1.4B. Matrix Effects which Interfere with Multiplex Immunoassays, (FIG. 1.1) Normal configuration of a multiplexed microsphere-based Luminex® sandwich immunoassay is shown as an example (microspheres labeled as “Assay Surface” to indicate this can be any type of solid-phase immunoassay, not just Luminex® microsphere-based multiplex immunoassays), Y-shaped structures are antibodies. The capture antibody (black) binds the analyte (e.g. biomarker), which is detected by another antibody (white with black outline) labeled with a fluorophore (F) (or other detectable label such as biotin). (FIG. 1.2) Low specificity heterophile antibodies (gray) in human serum matrices can bridge proteins on the assay surface (e.g. non-immune globulins or immunoglobulins, including the capture antibodies) to the detection antibodies yielding a false positive signal. (FIG. 1.3) Matrix-induced microsphere aggregation can also occur (e.g. via heterophile antibodies or other agents). (FIG. 1.4) Non-specific or even specific binding of any unintended matrix component to any component of the immunoassay can interfere, e.g. by (FIG. 1.4a) blocking binding of the analyte or (FIG. 1.4b) mediating background signals. Note that instead of the capture antibody on the assay surface, other assay capture agents can be used (not depicted in this figure). For example in the case of allergy testing for allergen-specific IgE (sIgE), the capture antibody is replaced with an allergen (antigen) on the assay surface, which could be a crude allergen extract (e.g. from a food) or a purified allergen component protein (e.g. Ara h 1 from peanuts). In this case, the analyte may itself be an antibody (e.g. sIgE for the allergy example) from a patient sample such a blood. Regardless of the assay capture agent, analyte, or detection method, the modes of the matrix effect are similar to as shown in this figure.

FIG. 2A-B. Example of Individual Steps for the Concentration and/or Purification of Analytes, Such as Biomarkers, Using Photocleavable Capture Agents (PC-Binding Agents): The PC-PURE Process. Not drawn to scale. (FIG. 2a) The analyte (e.g. biomarker) is the white triangle with the black outline. A substrate (well of a microtiter plate containing a micro-porous membrane, gel or film is depicted as an example) containing the attached photocleavable capture agent (“PC-Binding Agent” attached by a photocleavable linker [PC-linker]) is used for biomarker concentration and/or purification (the PC-PURE process). The PC-Binding Agent can for example be an aptamer (shown; multi-circle structure attached to plate surface); the PC-Binding Agent can for example also be an antibody, antigen or an engineered protein scaffold based binding agent (e.g. commercially available Affibodies®). The input sample volume and photo-release volume are shown (grayed areas in well). In this example, in addition to purification, the biomarker is also concentrated by photo-releasing in a smaller volume compared to the input sample volume. (FIG. 2b) In some cases, a downstream immunoassay can be performed following biomarker concentration and/or purification (following PC-PURE) as shown in steps 5-6 (prior steps are again PC-PURE, showing a generic microtiter plate as the PC-PURE substrate in this case). The immunoassay depicted is a multiplex Luminex® microsphere-based sandwich immunoassay (the Y-shaped structures are antibodies; gray antibody is the assay capture antibody and black is the assay detection antibody; reporter label not shown; the photocleaved PC-Binding Agent remains bound hut does not participate in the assay detection in this example; in other embodiments, the assay detection antibody is omitted and the photocleaved PC-Binding Agent instead used also for detection). Other immunoassay formats such as an immobilized-antigen format (antigen on assay surface binds an antibody biomarker) or a competitive inhibition format are possible. Assays other than immunoassays are also possible, such as mass spectrometry based biomarker detection assays.

FIG. 3A-B. IgE Capture and Photo-Release Efficiency of PC-Beads. (FIG. 3a) Loading the PC-Antibody to streptavidin agarose beads (preparing PC-Beads). PC-Biotin labeled anti-IgE antibody (PC-Antibody) was loaded onto streptavidin agarose beads to create the PC-Beads. Using a standard commercial colorirnetric ELISA, the amount of PC-Antibody was quantified in the “Input” (solution prior to adding to the streptavidin agarose beads) and “Depleted” fraction (solution after treatment with the streptavidin agarose beads). The Blank is the diluent buffer without PC-Antibody. The inset box is the ELISA standard curve using a 5-Parameter Logistic (5PL) curve fit (dotted lines are the 95% confidence bands). (FIG. 3b) Demonstrating the capture and photo-release capabilities of the PC-Beads. Digoxigenin labeled human IgE (Dig-IgE; the analyte) was captured on PC-Beads which contained the anti-IgE PC-Antibody. PC-Beads were then washed and illuminated with 365 nm UV light. Using a microsphere-based sandwich immunoassay, the amount of Dig-IgE was quantified in the “Input” (solution prior to adding to PC-Beads), “Depleted” fraction (solution after treatment with the PC-Beads) and “Photo-Released” fraction (solution after UV treatment of PC-Beads). For the immunoassay, an anti-digoxigenin capture antibody on the microspheres and an anti-IgE detection antibody were used (detection antibody binds different epitope than the PC-Antibody). The immunoassay results were interpolated from a Dig-IgE standard curve using a 5-Parameter Logistic (5PL) curve fit (see inset box; dotted lines are the 95% confidence bands; MFI=Median Fluorescence Intensity of the immunoassay). In the bar graph, the amount of Dig-IgE measured is expressed as a percent of the Input. For the “Sequential” method, photo-release was followed by applying the supernatant to the microspheres, whereas in the “Combined” method, photo-release was performed with the PC-Beads and microspheres together.

FIG. 4. Binding Capacity Estimate of PC-Beads. PC-Beads carrying the anti-IgE PC-Antibody were used to capture human IgE spiked at various concentrations into a buffer solution. Using a standard commercial colorimetric human IgE ELISA, the amount of IgE was quantified in the “Input” (solutions prior to adding to the PC-Beads) and “Depleted” fractions (solutions after treatment with the PC-Beads). The IgE in the post-capturing washes was also quantified and summed together with the results from the Depleted fractions; this is reported as the “Un-Captured” IgE amount. *The “Captured” IgE amount is calculated as the difference between the Input and the Un-Captured. The “Blank” corresponds to a Depleted fraction from a 0 μg/mL IgE Input. The inset box shows the ELISA standard curve with a 4-Parameter Logistic (4PL) curve fit.

FIG. 5. Elimination of the Matrix Effect from Multiplex In Vitro Allergy Assays (AllerBead) using PC-PURE. Multiplex AllerBead assays were performed with and without PC-Antibody based IgE pre-purification (“PC-PURE”). A model patient serum was used for this analysis which was known to be positive for milk allergen-specific IgE (sIgE) and negative for soy (determined a priori based on the gold-standard, FDA-cleared, non-multiplex ImmunoCAP® test). MFI=Median Fluorescence Intensity output of the Luminex® based AllerBead assays.

FIG. 6A-C. Performance Metrics of AllerBead with and without PC-PURE. Serum samples from 205 subjects presenting at Boston Children's Hospital with suspicion of or known food allergy were analyzed by the multiplex AllerBead assay against all eight food allergens under study. AllerBead was performed with and without PC-PURE pre-purification of patient IgE. Results from the gold-standard, FDA-cleared, non-multiplex ImmunoCAP® test for all eight foods were used as a reference and to determine true positives and negatives for allergen-specific IgE. (FIG. 6a) Signal-to-Noise of AllerBead. Signal-to-noise was calculated on a per-food basis as the average AllerBead result for ImmunoCAP®-positives (≥0.10 kIU_{A}/L) divided by the average AllerBead result of ImmunoCAP®-negatives (<0.10 kIU_{A}/L). (FIG. 6b) Pearson's r as a metric for ImmunoCAP®-correlation of the AllerBead assays. (FIG. 6c) Sensitivity of the AllerBead assays. *Sensitivity was defined as the percent of ImmunoCAP®-positives detected in the range of the maximum measurable by ImmunoCAP® (100 kIU_{A}/L) down to the cutoffs for 95% negative predictive value (NPV) for determining clinical allergy. 95% NPV cutoffs ranged from 0.35 kIU_{A}/L to 5 kIU_{A}/L depending on the food. 95% NPV cutoffs were based on prior literature reports using ImmunoCAP® or equivalent assays in comparison to food challenge (see Specification for references); if 95% NPV was not reached m those studies, cutoff for best achieved NPV was used (see Table 1 for cutoffs and NPVs). Note NPV cutoffs have not been published for all eight foods under study and thus Shrimp and Cashew are omitted. AllerBead sensitivity for peanut is a composite of peanut extract and Ara h 8, and for milk, a composite of milk extract and lactalbumin (Bos d 4).

FIG. 7. Example ImmunoCAP®-Correlation of AllerBead with and without PC-PURE. Regression analysis of the multiplex AllerBead assays with and without PC-PURE purification of IgE, compared to the gold-standard, FDA-cleared, non-multiplex ImmunoCAP® test (for the tree nut cashew) for all 205 Boston Children's Hospital patients. Note that AllerBead results were converted to kIU_{A}/L by heterologous interpolation from a standard curve (5 points; R² of linear regression=0.99) comprised of purified IgE from the serum of patients with various known amounts of sIgE (based on ImmunoCAP® testing). Pearson's r and slope of the regression lines are provided. Pearson's r for all foods are shown in FIG. 6b.

Table 1. AllerBead with PC-PURE in Reference to ImmunoCAP® on 205 Fully Annotated Boston Children's Hospital Serum Samples.

FIG. 8. Concentrating Patient IgE with PC-PURE; Increased Low-End Sensitivity for sIgE. The PC-PURE method was used to concentrate IgE from 46 food allergy samples followed by analysis on the multiplex AllerBead assay. To achieve the concentrating effect, the input sample volume for the PC-PURE step was 500 μL and the photo-release volume was 100 μL (“5×”), which was input into the multiplex allergen immunoassay. This was compared to AllerBead performed without concentrating (100 μL input and photo-release volumes). Sensitivity (percent of ImmunoCAP®-positives detected by AllerBead) was assessed in the low-end of the ImmunoCAP® scale, between 0.35 kIU_{A}/L and 5 kIU_{A}/L.

FIG. 9A-C. Biotin-Phycoerythrm (Biotin-PE) Binding Capacity of Various NeutrAvidin and Streptavidin Coated Microtiter Plates. (FIG. 9a) NeutrAvidin-coated nitrocellulose membrane-bottom plates (“Nitrocellulose NeutrAvidin”) versus commercially available solid polystyrene streptavidin-coated high capacity plates (“Thermo Streptavidin”). 1 hr Biotin-PE binding time. (FIG. 9b) NeutrAvidin-coated nitrocellulose membrane-bottom plates (“Nitrocellulose NeutrAvidin”) versus NeutrAvidin-coated PVDF membrane-bottom plates (“PVDF NeutrAvidin”). 1 hr Biotin-PE binding time. (FIG. 9c) Overnight versus 1 hr Biotin-PE binding time on NeutrAvidin-coated nitrocellulose membrane-bottom plates (“Nitrocellulose NeutrAvidin”) and commercially available solid polystyrene streptavidin-coated high capacity plates (“Thermo Streptavidin”). *Specific binding was calculated by correcting for non-specific binding, by subtracting out the binding occurring on the negative control wells which lacked a NeutrAvidin coating (specific binding could not be calculated with Thermo Streptavidin plates since wells produced in the same manner but lacking the streptavidin coating were not available).

FIG. 10A-B. Comparison of Various PC-PURE Methods Followed by Multiplex Microsphere-Based Immunoassay of Allergen-Specific IgE (the AllerBead assay). IgE was PC-PURE purified from serum samples followed by measurement of allergen-specific IgE (sIgE) using the multiplex AllerBead assay. sIgE positivity or negativity was also confirmed by analysis of the same serum samples using the FDA-cleared, gold-standard, non-multiplex ImmunoCAP® assay. (FIG. 10a) PC-PURE was compared using an anti-IgE photocleavable antibody (PC-Antibody) on streptavidin agarose beads (PC-Beads), on a NeutrAvidin-coated nitrocellulose membrane-bottom microtiter plate (Nitrocellulose PC-Plate) and on a commercial Thermo Scientific high capacity solid polystyrene streptavidin-coated microtiter plate (Thermo PC-Plate). Analysis was of 24 serum, samples and 7 food allergens (peanut, shrimp, cashew, egg white, cod, wheat and soy). AllerBead signal-to-noise ratio was calculated and averaged for all ImmunoCAP®-positive data points within each food allergen. (FIG. 10b) PC-PURE was compared using an anti-IgE photocleavable antibody (PC-Antibody) on a NeutrAvidin-coated nitrocellulose membrane-bottom microtiter plate (Nitrocellulose PC-Plate) and on a NeutrAvidin-coated PVDF membrane-bottom microtiter plate (PVDF PC-Plate). Analysis was of 16 serum samples and 8 food allergens (peanut, milk, shrimp, cashew, egg white, cod, wheat and soy). A regression plot of the MFI (Median Fluorescence Intensity), the raw output of the AllerBead assay, is shown for all data points (all samples and all food allergens [food extracts only]).

Table 2. Pearson's Correlation (r Value) with ImmunoCAP® of Various PC-PURE IgE Purification Methods Followed by Multiplex Microsphere-Based Immunoassay of Allergen-Specific IgE (the AllerBead assay). PC-Beads (porous agarose beads containing the PC-Antibody), a Nitrocellulose PC-Plate (porous nitrocellulose membrane-bottom microtiter plate containing the PC-Antibody) and a Thermo PC-Plate (solid polystyrene microtiter plate containing the PC-Antibody) were used for the PC-PURE steps. 24 serum samples were analyzed. The number of ImmunoCAP®-positive and negative data points is also given.

FIG. 11A-B. PC-PURE Using Custom Cast Nitrocellulose Membranes in Solid Microtiter Plates vs. Commercial Nitrocellulose-Bottom Microtiter Filter Plates; Application to the AllerBead Assay: (FIG. 11a) Image showing nitrocellulose membranes cast into solid, glass-coated, polypropylene microtiter plates by depositing nitrocellulose (NC) solutions and drying. Two concentrations of nitrocellulose solutions were used for casting, 85 mg/mL and 34 mg/mL. (FIG. 11b) The custom cast nitrocellulose plates (data shown for 34 mg/mL condition) and commercially available nitrocellulose membrane-bottom filter plates were coated with NeutrAvidin and then the anti-IgE PC-Antibody, referred to as “Custom Nitrocellulose PC-Plates” and “Commercial Nitrocellulose PC-Plates”, respectively. The plates were then used for PC-PURE of a monoclonal humanized chimeric IgE anti-Der p 2 antibody (“Anti-Der P2 IgE”) which was spiked into a buffer at various concentrations. The AllerBead assay followed. MFI=Median Fluorescence Intensity (raw output of the AllerBead assay).

FIG. 12A-B. Comparison of Finger-Stick Capillary Serum to Venous Draw, and Room Temperature Serum Storage to Storage Frozen: PC-PURE Followed by the AllerBead Assay. PC-PURE IgE purification from serum using PC-Beads was followed by the multiplex AllerBead assay for quantification of allergen-specific IgE (sIgE) to various food allergens (see Example 4 for allergens). Regression plots of the Median Fluorescence Intensity (MFI), the raw output of the AllerBead assay, were made comparing the following conditions (data points for all samples and all food allergens [food extracts only]are plotted): (FIG. 12a) Matched finger-stick derived capillary serum versus venous derived serum from the same patients (8 samples and 8 food allergen extracts plotted). (FIG. 12b) Room temperature stored venous derived serum (10 days) versus aliquots of the same samples stored frozen (14 samples and 8 food allergen extracts plotted).

FIG. 13A-B. PC-PURE of Cancer Biomarkers (Target Proteins) using Photocleavable Aptamers (PC-Aptamers): Downstream Sandwich Immunoassay on a Luminex® Multiplex-Compatible Microsphere-Based Platform. (FIG. 13a) Four cancer biomarkers (VEGFR, HGFR, EGFR and AKT2) each in plain buffer were subjected to PC-Aptamer based PC-PURE (using microtiter plates). The “Input” sample is the solution prior to isolation on the PC-Aptamer coated microtiter plates. The “Photo-Release” fraction is the solution after elution from the PC-Aptamer coated microtiter plates using UV light treatment. The “Input” samples as well as the “Photo-Release” sample fractions were measured by a sandwich immunoassay on the multiplex Luminex® microsphere-based platform (the PC-Aptamer is used only for PC-PURE, and although present, does not participate in the Luminex® immunoassay that follows). “Blank” indicates where the initial Input lacked the biomarker and in all other cases the initial Input contained the biomarker. (FIG. 13b) The VEGFR protein biomarker was spiked into plain buffer and serum at various concentrations. PC-PURE with a PC-Aptamer (on agarose beads in this case) was used to purify and concentrate VEGFR followed by a Luminex® microsphere-based sandwich immunoassay. This was compared to “Standard Luminex®” analysis (direct immunoassay of the crude serum without PC-PURE), MFI=Median Fluorescence Intensity, the raw output of the Luminex® immunoassay.

FIG. 14A-C. Dual-Labeled Photocleavable & Fluorescent Binding Agents: Integrating PC-PURE with Downstream Detection. (FIG. 4a) The dual-labeled PCB-PE-Anti-TIMP antibody on microtiter plates was used for isolation of the TIMP protein. Free TIMP was quantified in the “Input” solution (TIMP solution prior to isolation) and “Depleted” fraction (TIMP solution after isolation). Plus or minus “Antibody” indicates whether or not the dual-labeled PCB-PE-Anti-TIMP antibody was present on the microtiter plate used for TIMP isolation. The “Blank” is plain diluent without TIMP and not subjected to the isolation procedure. (FIG. 14b) The dual-labeled PCB-PE-Anti-TIMP antibody was used for detection of the TIMP protein in a Luminex® microsphere-based multiplex-compatible sandwich immunoassay (no PC-PURE in this case). (1.) The standard Luminex® detection system which uses a biotin-anti-TIMP antibody followed by a fluorescent streptavidin-PE conjugate was compared to (2.) the dual-labeled PCB-PE-Anti-TIMP antibody alone as the detection reagent. “+TIMP” indicates samples containing TIMP and “Blank” indicates samples without. The Luminex® assay signal is expressed in MFI, raw Median Fluorescence Intensity. (FIG. 14c) TIMP was subjected to PC-PURE using the dual-labeled PCB-PE-Anti-TIMP antibody on agarose beads. The “Input” sample is the solution prior to isolation by PC-PURE. The “Photo-Release” step of PC-PURE was performed with and without the necessary UV treatment (plus or minus “UV”). The “Input” sample and “Photo-Release” sample fractions were measured by sandwich immunoassay on the multiplex Luminex® platform (where the photocleaved PCB-PE-Anti-TIMP antibody also serves as the detection reagent). “+TIMP” indicates where the initial Input contained TIMP and “Blank” indicates where TIMP was omitted from the initial Input. PCB=Photocleavable Biotin; PE=Phycoerythrin.

FIG. 15A-C. Biotin-Phycoerythrin (Biotin-PE) Binding of Nitrocellulose Plates Directly and Indirectly Coated with NeutrAvidin: Comparison to Thermo Streptavidin Plates. Nitrocellulose membrane-bottom microtiter plates were either directly coated with NeutrAvidin by passive adsorption (“Direct NeutrAvidin Nitrocellulose”) or indirectly coated by passively adsorbing Biotin-BSA first and then attaching (tetrameric) NeutrAvidin (“Indirect NeutrAvidin Nitrocellulose”). Biotin-PE binding was then assessed as a function of the amount of Biotin-PE input per well (note that a constant volume of 150 μL/well of Biotin-PE input was used, therefore, the concentration of Biotin-PE was variable). Comparisons were also made with commercially available high capacity streptavidin coated solid microtiter plates (“Thermo Streptavidin Plates”). Bound Biotin-PE per well was plotted versus the input amount. (FIG. 15a) Bar graph showing the full range of Biotin-PE inputs. (FIG. 15b) Line plot showing the mid- to low-range of Biotin-PE inputs. (FIG. 15c) scatter plot showing the linear range of bound Biotin-PE as a function of the input amount. Dotted lines are the best fit linear regression lines (R² values, not shown, were >0.99 in all cases).

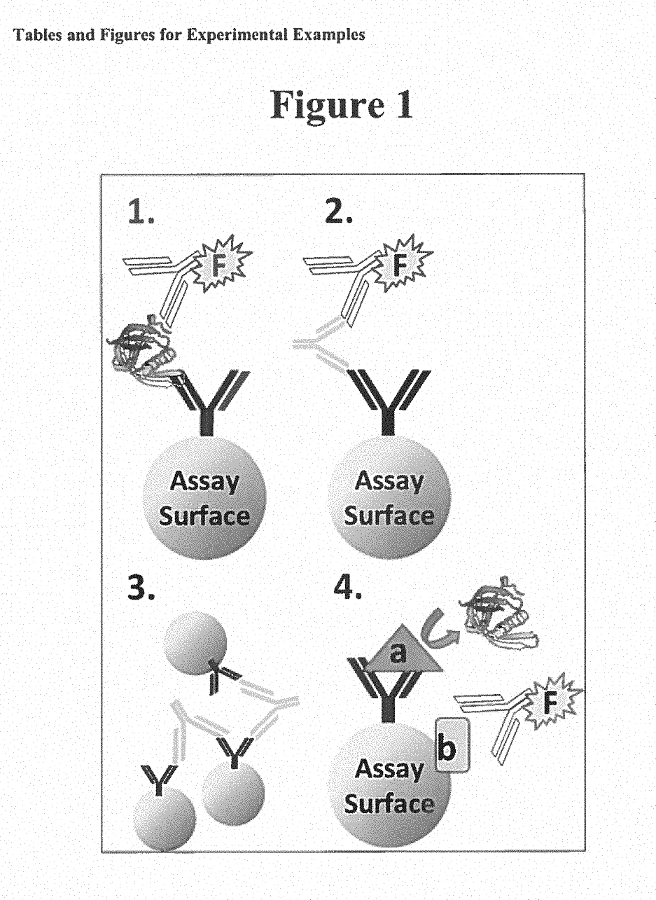

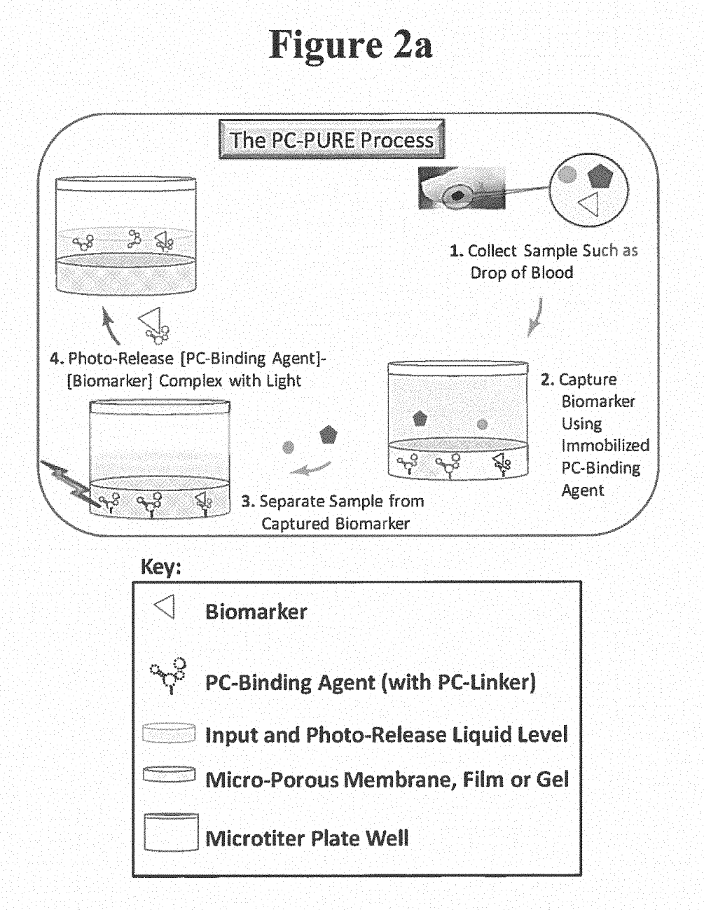

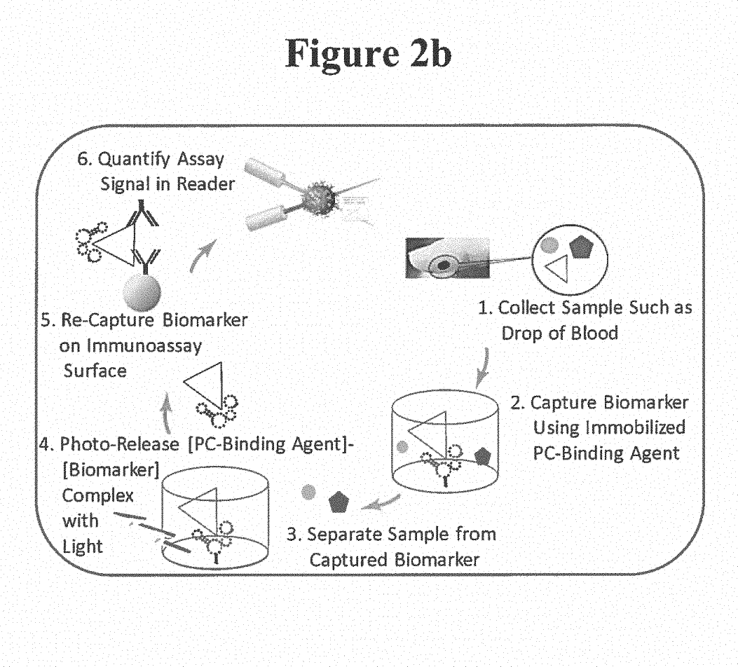

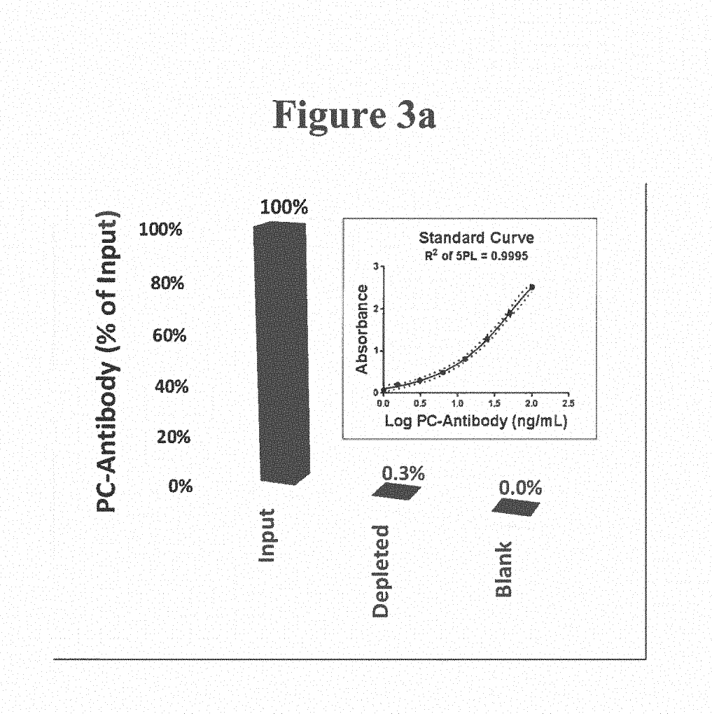

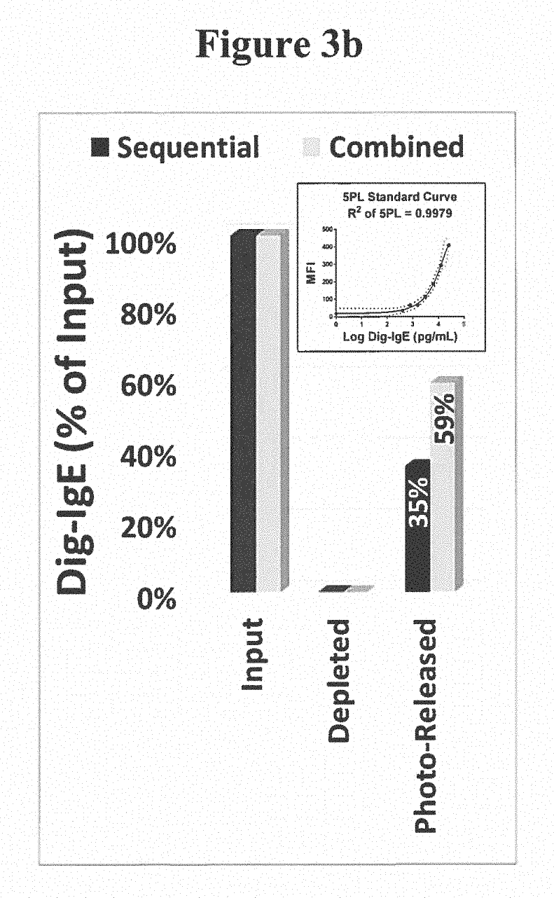

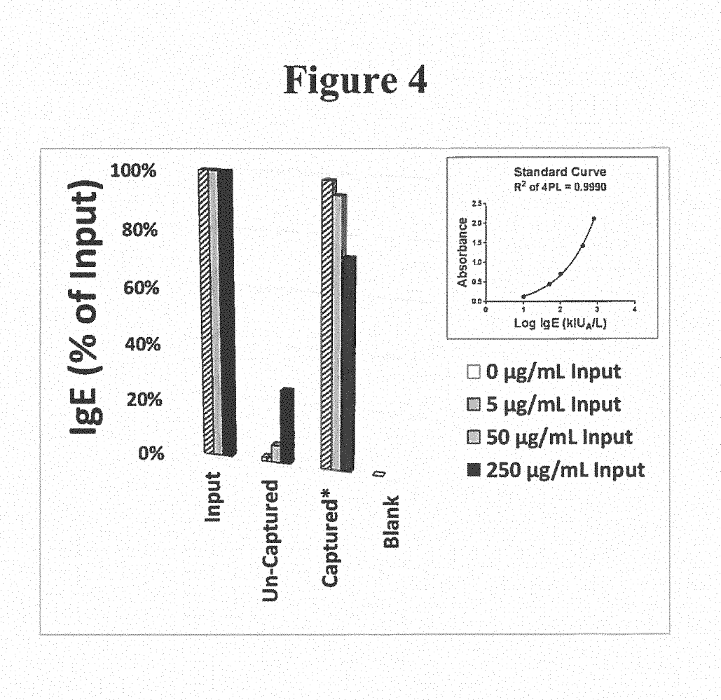

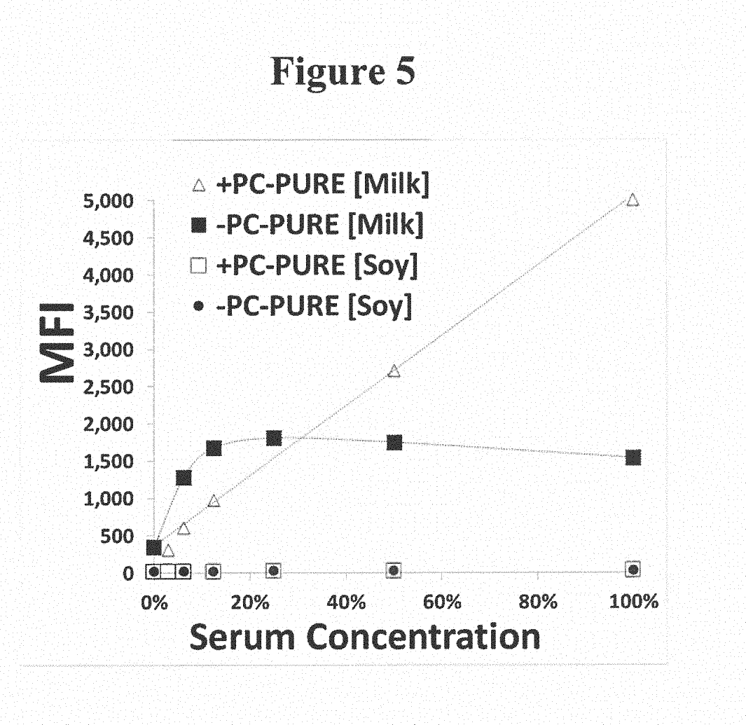

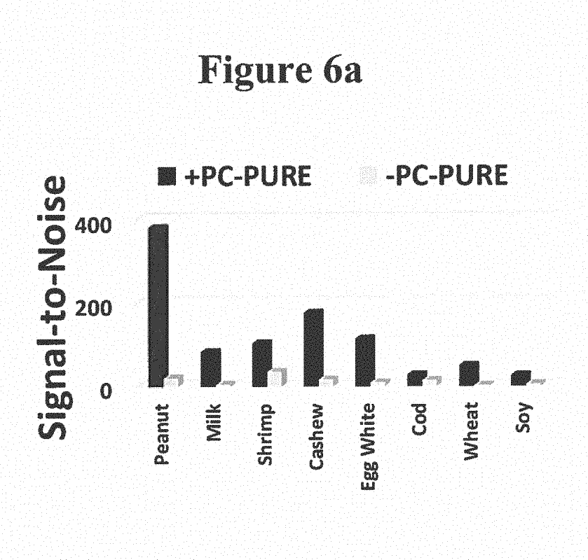

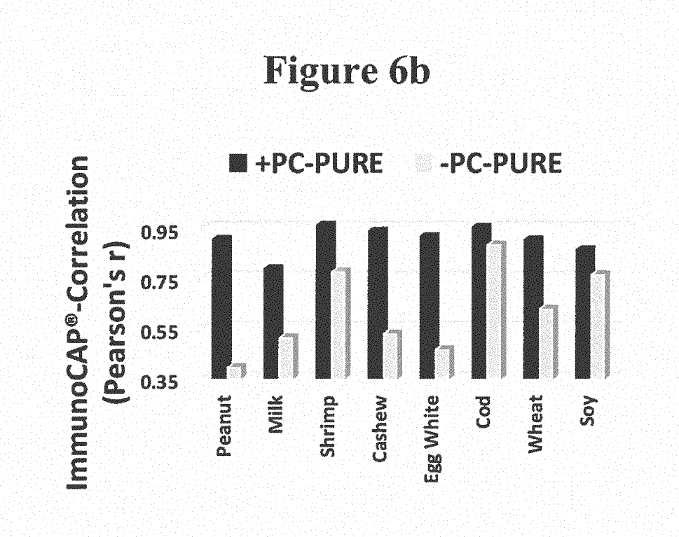

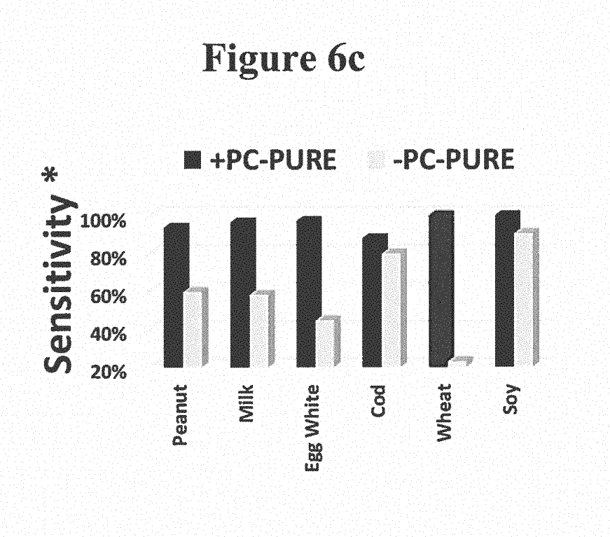

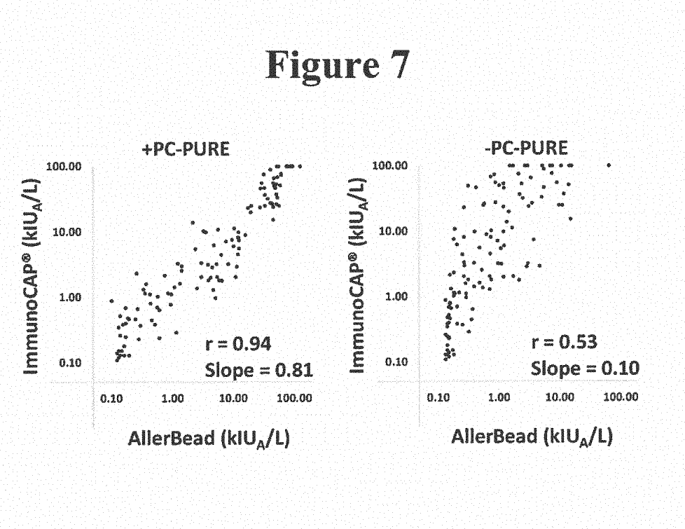

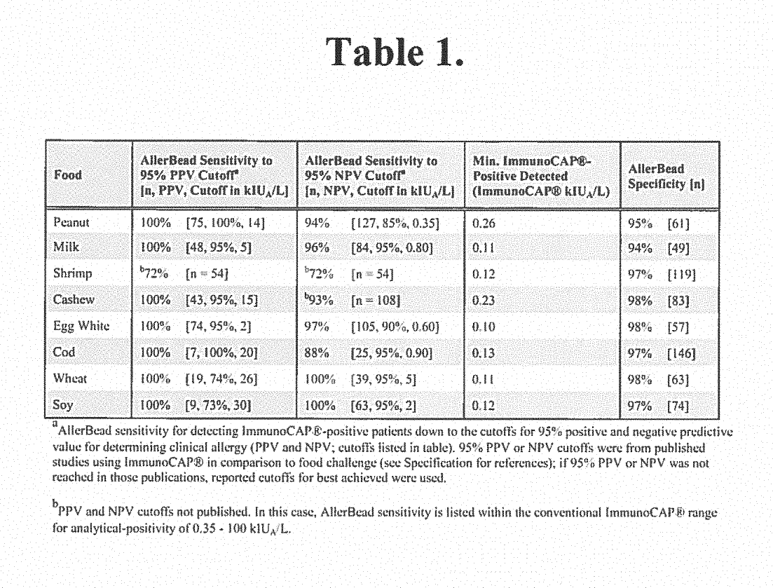

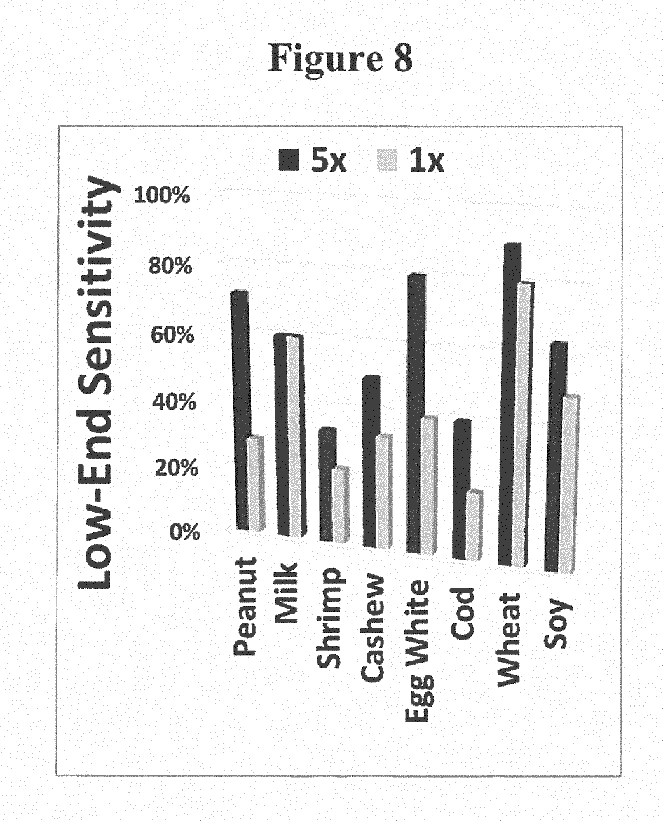

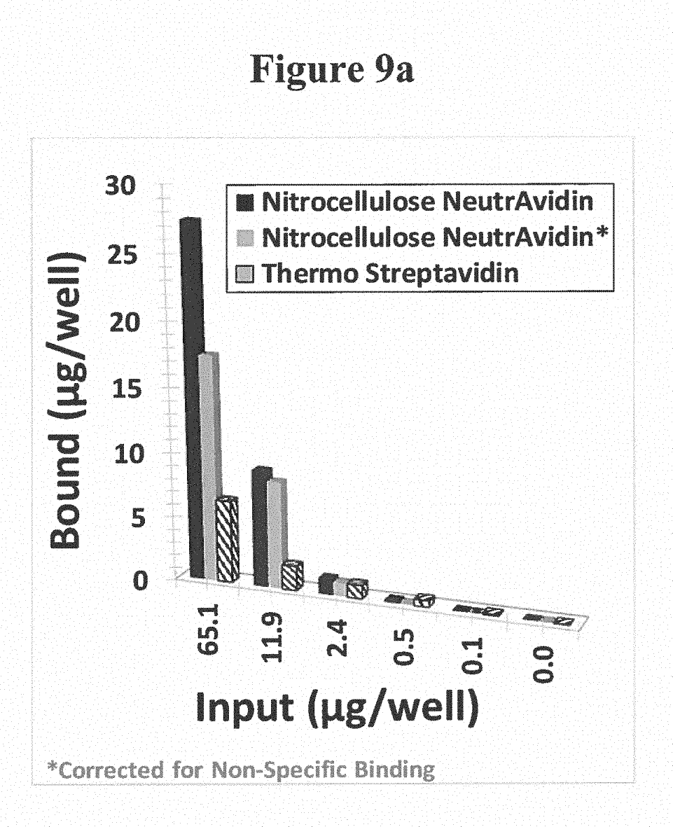

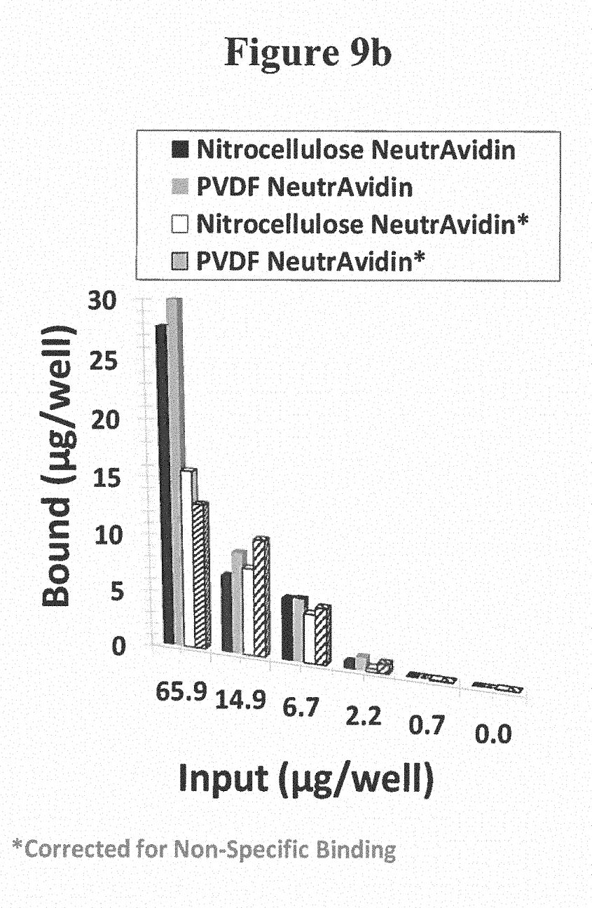

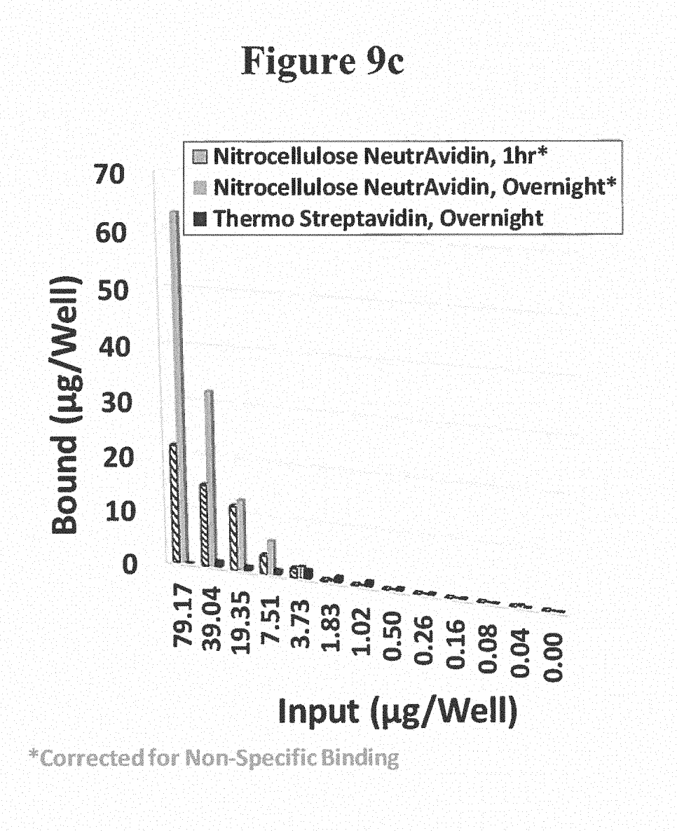

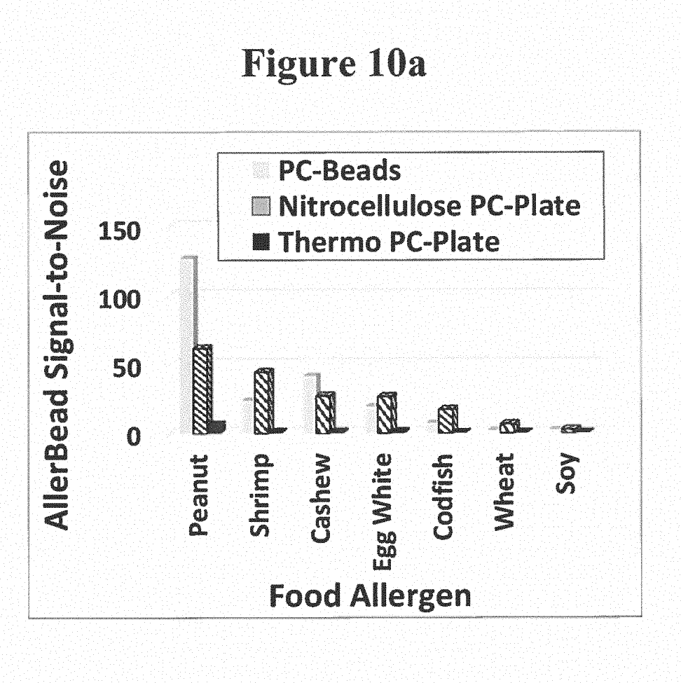

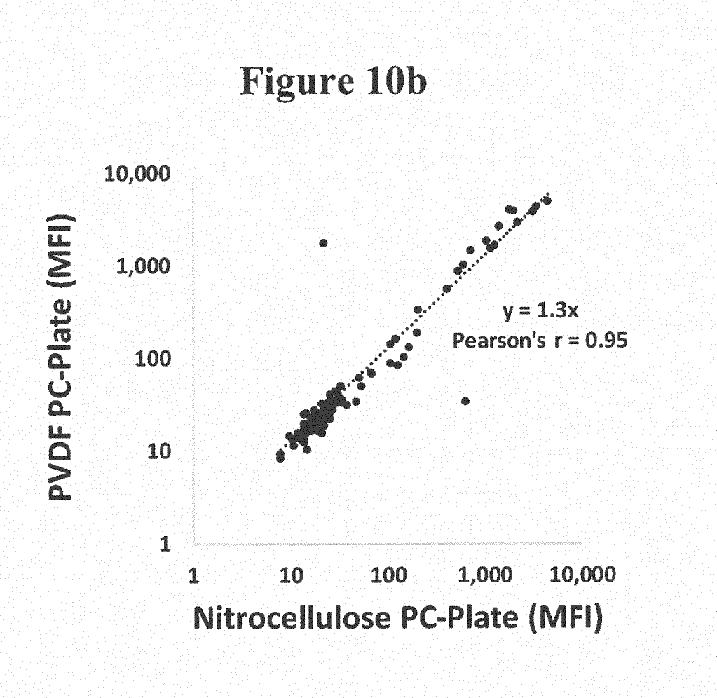

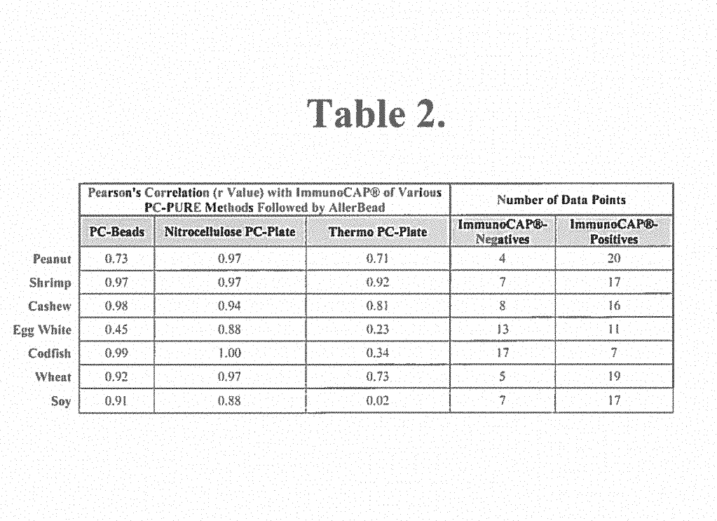

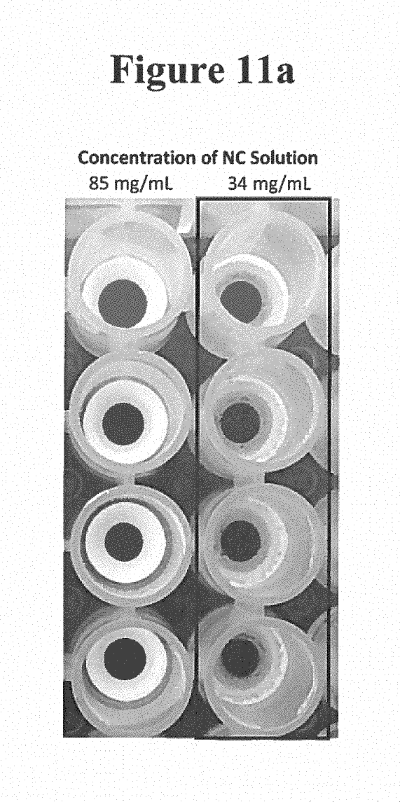

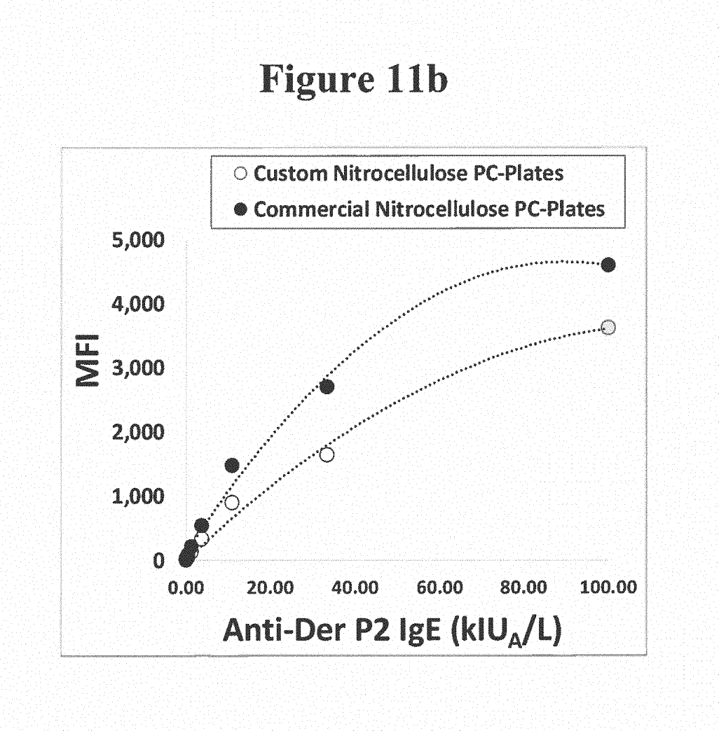

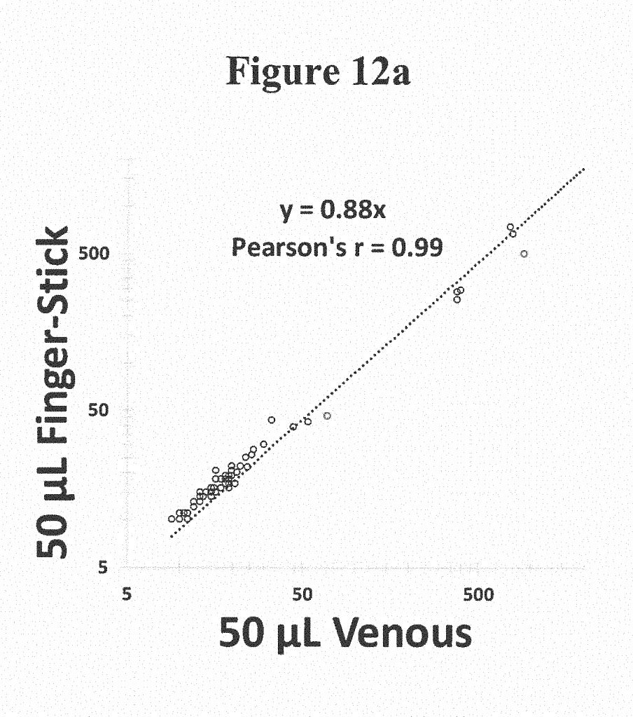

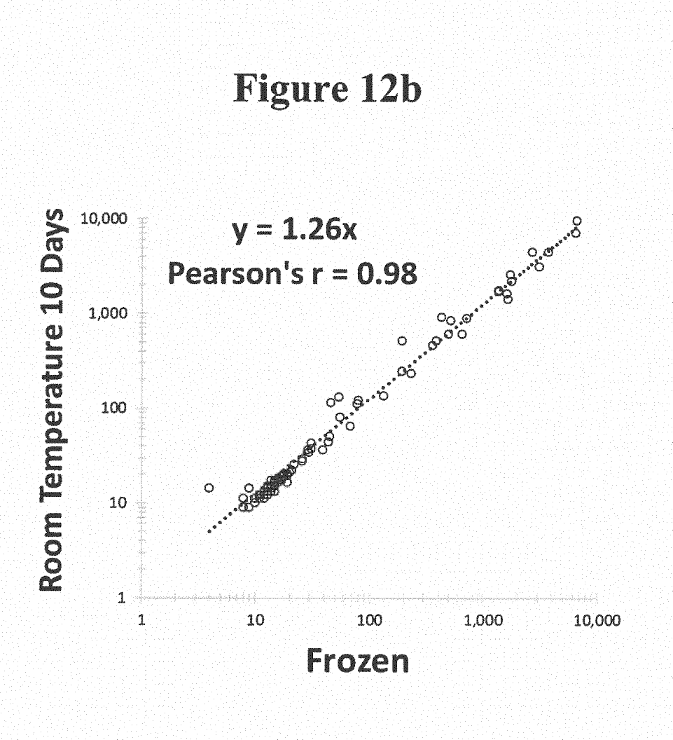

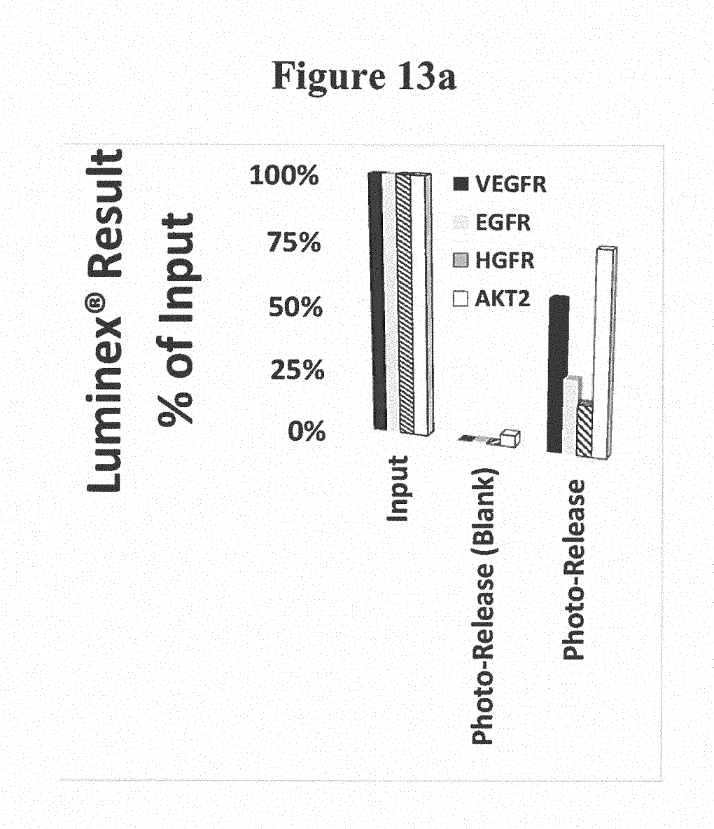

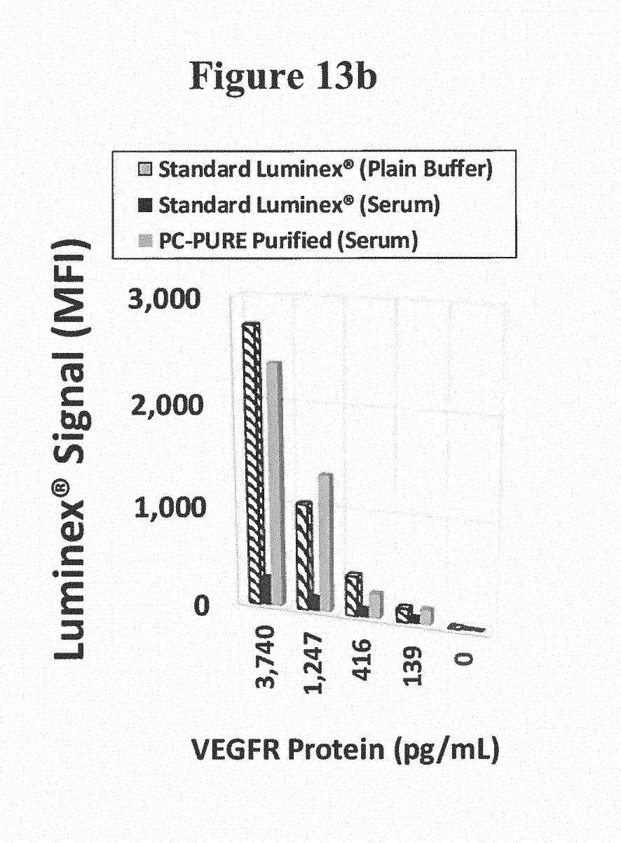

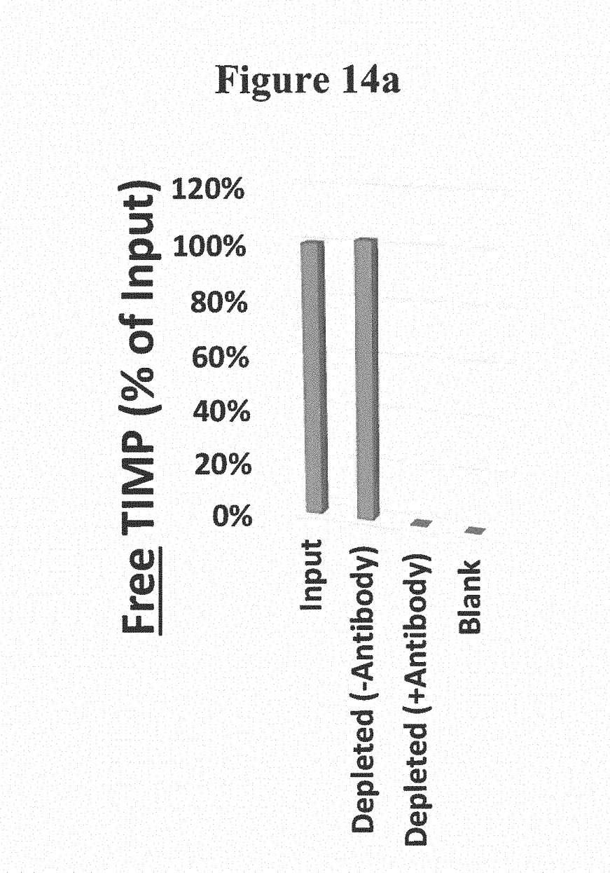

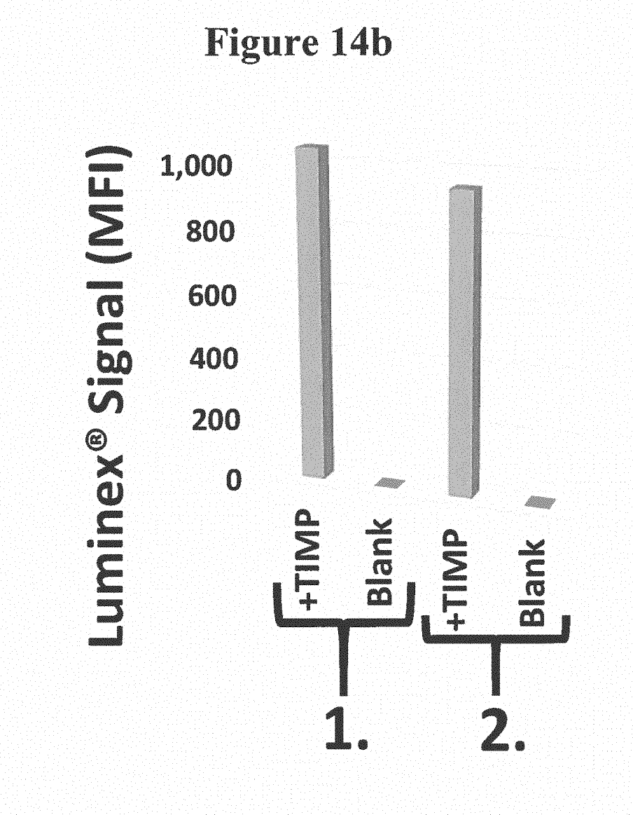

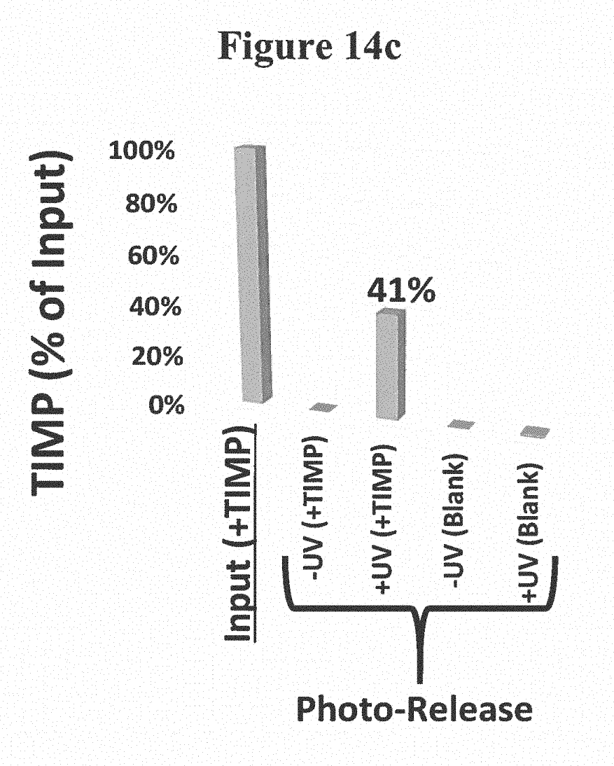

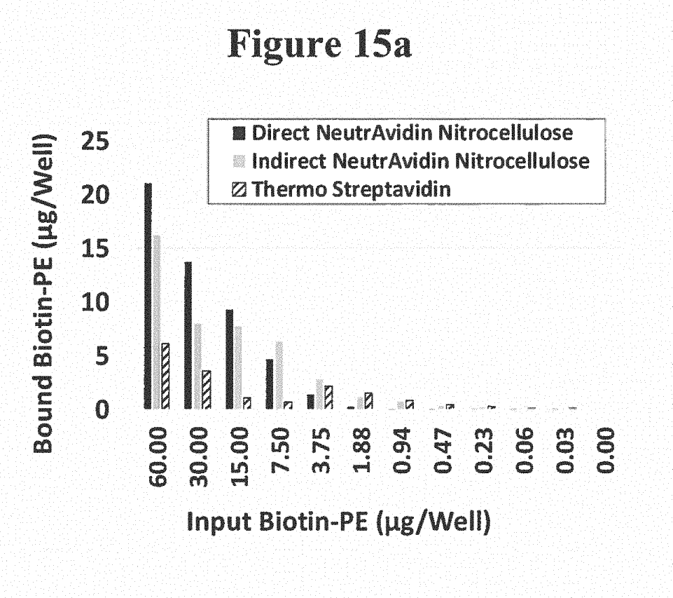

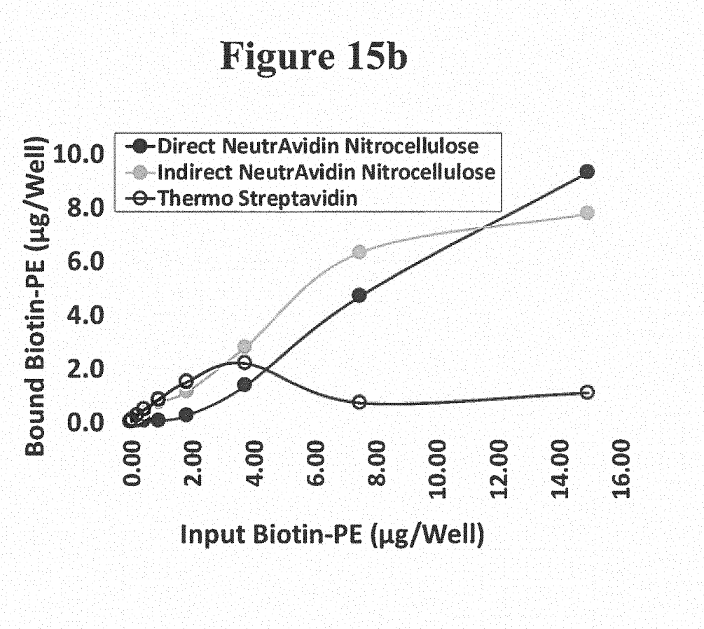

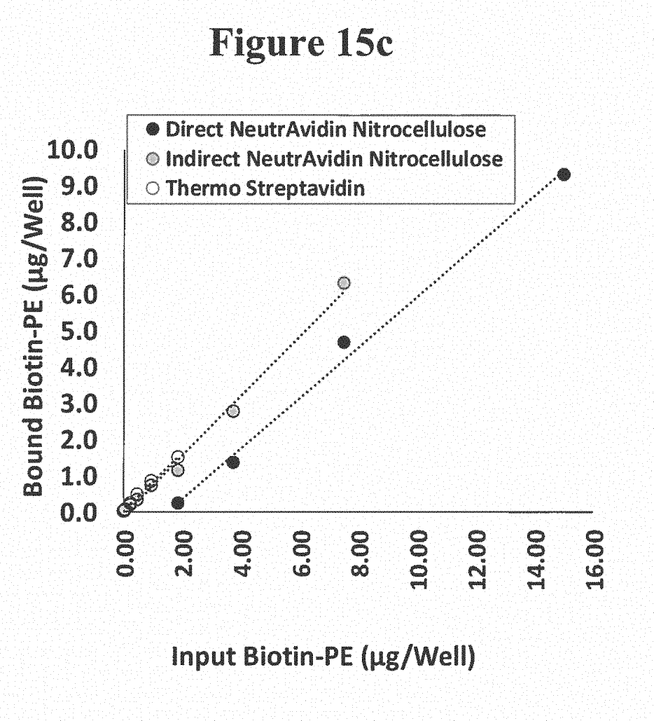

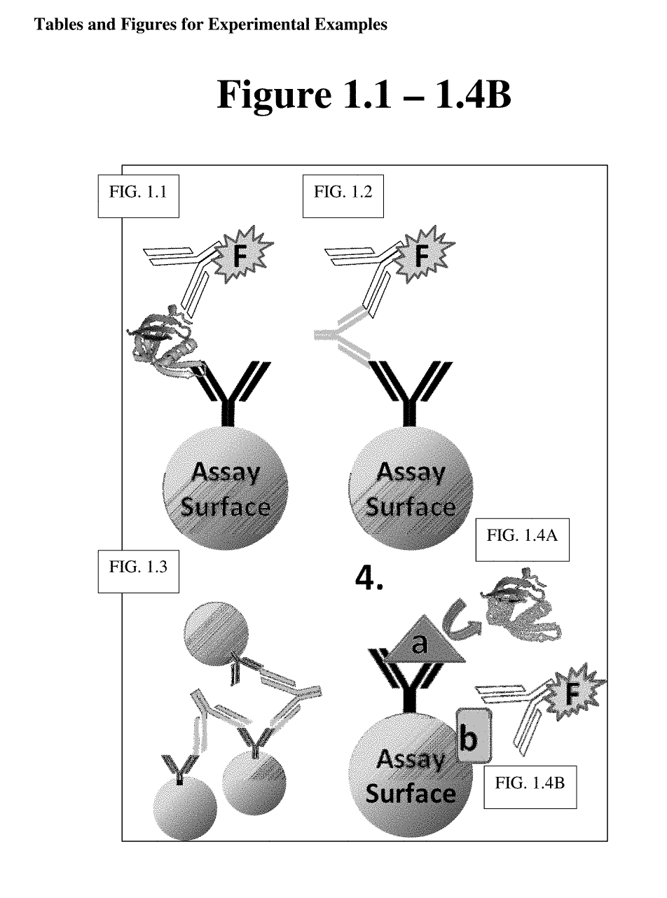

What is claimed is:

1. A composition for the photocleavage based concentration and purification of analytes from liquid samples, comprising:
a. a microtiter plate having wells, wherein at least a portion of the interior surface of said wells comprises a micro-porous membrane; and
b. at least one of said wells in said microtiter plate having binding agents directly or indirectly attached by a photocleavable linker to said micro-porous membrane; and
c. wherein the at least one of said wells contains a liquid sample within, wherein said liquid sample comprises analyte molecules, and wherein said liquid sample contacts 100% of the top surface of said micro-porous membrane; and
d. wherein at least a portion of said binding agents attached to said well containing said liquid sample are bound to at least a portion of said analyte molecules from said liquid sample.

2. The composition of claim 1, wherein said micro-porous membrane comprises nitrocellulose and other cellulose esters.

3. The composition of claim 1, wherein said micro-porous membrane comprises PVDF.

4. The composition of claim 1, wherein said microtiter plate is a microtiter filter plate having said micro-porous membrane as the well bottoms.

5. The composition of claim 1, wherein said microtiter plate is a solid-bottom microtiter plate having said micro-porous membrane cast onto the well bottoms.

6. The composition of claim 1, wherein said binding agent is selected from the group consisting of antibodies or fragments thereof, aptamers and engineered protein scaffold based binding agents.

7. The composition of claim 1, wherein said binding agent is also conjugated to a detectable label.

8. The composition of claim 7, wherein said detectable label is a fluorescent label.

9. The composition of claim 1, wherein said micro-porous membrane is coated with avidin, streptavidin or NeutrAvidin.

10. The composition of claim 1, wherein said photocleavable linker is photocleavable biotin.

11. The composition of claim 1, wherein said photocleavable linker comprises 2-nitrobenzyl or 1-(2-nitrophenyl)-ethyl moieties.

12. A method for the photocleavage based concentration and purification of analytes from liquid samples, comprising:
a. providing
i. a microtiter plate having wells, wherein at least a portion of the interior surface of said wells comprises a micro-porous membrane; and
ii. at least one of said wells in said microtiter plate having binding agents directly or indirectly attached by a photocleavable linker to said micro-porous membrane; and
iii. a liquid sample containing analyte molecules capable of binding to said binding agents; and
iv. a source of electromagnetic radiation; and
v. an uptake liquid.
b. depositing at least a portion of said liquid sample into the at least one of said wells having said binding agents, wherein said liquid sample contacts 100% of the top surface of said micro-porous membrane, under conditions such that at least a portion of said analyte molecules bind to at least a portion of said binding agents; and
c. illuminating at least a portion of said binding agents having said bound analyte molecules with radiation from said radiation source under conditions such that at least a portion of said binding agents are photocleaved into said uptake liquid, wherein the concentration or purity of said analyte in said uptake liquid is greater than that in said liquid sample from step a. iii.

13. The method of claim 12, wherein said micro-porous membrane comprises nitrocellulose and other cellulose esters.

14. The method of claim 12, wherein said micro-porous membrane comprises PVDF.

15. The method of claim 12, wherein said microtiter plate is a microtiter filter plate having said micro-porous membrane as the well bottoms.

16. The method of claim 12, wherein said microtiter plate is a solid-bottom microtiter plate having said micro-porous membrane cast onto the well bottoms.

17. The method of claim 12, wherein said binding agent is selected from the group consisting of antibodies or fragments thereof, aptamers and engineered protein scaffold based binding agents.

18. The method of claim 12, wherein said binding agent is also conjugated to a detectable label.

19. The method of claim 18, wherein said detectable label is a fluorescent label.

20. The method of claim 12, wherein said micro-porous membrane is coated with avidin, streptavidin or NeutrAvidin.

21. The method of claim 12, wherein said photocleavable linker is photocleavable biotin.

22. The method of claim 12, wherein said photocleavable linker comprises 2-nitrobenzyl or 1-(2-nitrophenyl)-ethyl moieties.

23. A method for the photocleavage based concentration and purification of analytes from liquid samples, comprising:
a. providing
i. a microtiter plate having wells, wherein at least a portion of the interior surface of said wells comprises a micro-porous membrane; and
ii. at least one of said wells in said microtiter plate having binding agents directly or indirectly attached by a photocleavable linker to said micro-porous membrane; and
iii. a liquid sample containing analyte molecules capable of binding to said binding agents; and
iv. a source of electromagnetic radiation; and
v. an uptake liquid comprising a plurality of beads, microspheres or particles capable of binding to said analyte molecules.
b. depositing at least a portion of said liquid sample into the at least one of said wells having said binding agents, wherein said liquid sample contacts 100% of the top surface of said micro-porous membrane, under conditions such that at least a portion of said analyte molecules bind to at least a portion of said binding agents; and
c. illuminating at least a portion of said binding agents having said bound analyte with radiation from said radiation source under conditions such that at least a portion of said binding agents are photocleaved into said uptake liquid comprising a plurality of beads, microspheres or particles, wherein the concentration or purity of said analyte in said uptake liquid is greater than that in said liquid sample from step a. iii.; and
d. after said photocleaving, capturing at least a portion of said analyte molecules in said uptake liquid on at least a portion of said beads, microspheres or particles.

24. The method of claim 23, wherein said micro-porous membrane comprises nitrocellulose and other cellulose esters.

25. The method of claim 23, wherein said micro-porous membrane comprises PVDF.

26. The method of claim 23, wherein said microtiter plate is a microtiter filter plate having said micro-porous membrane as the well bottoms.

27. The method of claim 23, wherein said microtiter plate is a solid-bottom microtiter plate having said micro-porous membrane cast onto the well bottoms.

28. The method of claim 23, wherein said binding agent is selected from the group consisting of antibodies or fragments thereof, aptamers and engineered protein scaffold based binding agents.

29. The method of claim 23, wherein said binding agent is also conjugated to a detectable label.

30. The method of claim 29, wherein said detectable label is a fluorescent label.

31. The method of claim 23, wherein said micro-porous membrane is coated with avidin, streptavidin or NeutrAvidin.

32. The method of claim 23, wherein said photocleavable linker is photocleavable biotin.

33. The method of claim 23, wherein said photocleavable linker comprises 2-nitrobenzyl or 1-(2-nitrophenyl)-ethyl moieties.
